(19) United States
(12) Patent Application Publication
(10) Pub. No.: US 2018/0275084 A1
(43) Pub. Date: Sep. 27, 2018

(54) SENSOR

(71) Applicants: KABUSHIKI KAISHA TOSHIBA, Tokyo (JP); TOSHIBA ELECTRONIC DEVICES & STORAGE CORPORATION, Tokyo (JP)

(72) Inventors: Tatsuro SAITO, Kawasaki Kanagawa (JP); Yoshiaki SUGIZAKI, Fujisawa Kanagawa (JP); Atsunobu ISOBAYASHI, Yokohama Kanagawa (JP); Akihiro KAJITA, Yokohama Kanagawa (JP)

(73) Assignees: KABUSHIKI KAISHA TOSHIBA, Tokyo (JP); TOSHIBA ELECTRONIC DEVICES & STORAGE CORPORATION, Tokyo (JP)

(21) Appl. No.: 15/910,815

(22) Filed: Mar. 2, 2018

(30) Foreign Application Priority Data
Mar. 24, 2017 (JP) 2017-059813
Mar. 1, 2018 (JP) 2018-036536

(51) Int. Cl. G01N 27/12; C12Q 1/00; G01N 33/551; G01N 33/543

(52) U.S. Cl. CPC G01N 27/125; C12Q 1/001; G01N 27/128; G01N 33/54373; G01N 33/551

(57) ABSTRACT

According to one embodiment, a sensor includes a graphene film and at least two electrodes. The graphene film has an opening. The opening dominantly has either a zigzag edge or an armchair edge. The two electrodes electrically contact the graphene film, for reading a change in electric characteristics of the graphene film due to coaction with an object to be detected.

### CROSS-REFERENCE TO RELATED APPLICATIONS

This application is based upon and claims the benefit of priority from Japanese Patent Application No.2017-059813, filed on Mar. 24, 2017, and Japanese Patent Application No.2018-036536, filed on Mar. 1, 2018; the entire contents of all of which are incorporated herein by reference.

### FIELD

Embodiments described herein relate generally to a sensor.

### BACKGROUND

Graphene films exhibit large changes in electric characteristics (high sensitivity) in the bonding, adsorption, or proximity of atoms and molecules on the surface thereof. With such graphene films, application is particularly anticipated in the medical field, such as, for example, ion sensors, enzyme sensors, DNA sensors, antigen/antibody sensors, protein sensors, breath sensors, gas sensors.

### BRIEF DESCRIPTION OF THE DRAWINGS

FIGS. 1A and 1B are schematic diagrams of a sensor of an embodiment;

FIG. 2A is a schematic plan view of a sensor of an embodiment, and FIG. 2B is a schematic cross-sectional view of the sensor of the embodiment;

FIG. 3 to FIG. 5 are lattice structural views of a graphene film of an embodiment;

FIG. 6A is a lattice structural view of a graphene film of an embodiment, and FIG. 6B is a schematic cross-sectional view of a sensor of an embodiment;

FIG. 7 to FIG. 10 are lattice structural views of a graphene film of an embodiment;

FIG. 11A is a schematic diagram for describing a zigzag edge of a graphene film, and FIG. 11B is a schematic diagram for describing an armchair edge of a graphene film;

FIG. 12A is an arrangement diagram of two sublattices on a zigzag edge of a graphene film, and FIG. 12B is an arrangement diagram of two sublattices on an armchair edge of a graphene film;

FIG. 13 to FIG. 16 are schematic plan views of a graphene film of an embodiment;

FIG. 17A to FIG. 18 are schematic sectional views showing a method for manufacturing a sensor of an embodiment;

FIG. 19 is a schematic cross-sectional view of a sensor of another embodiment;

FIG. 20 is a schematic view showing a method for manufacturing a sensor of another embodiment;

FIG. 21A to FIG. 25B are schematic sectional views showing a method for manufacturing a sensor of another embodiment;

FIG. 26 to FIG. 28 are schematic views showing a method for manufacturing a sensor of another embodiment;

FIGS. 29A to 29C are schematic cross-sectional views of a substance recognition device of an embodiment;

FIG. 30 is a schematic cross-sectional view of a sensor of an embodiment;

FIG. 31A to FIG. 32B are graphs illustrated an example of Id-Vg characteristics in a sensor of an embodiment;

FIG. 33 is a schematic cross-sectional view of a reference element;

FIG. 34 is a diagram illustrated Id-Vg shift before and after measuring an object to be detected in each of a sensor of an embodiment and a reference element;

FIG. 35 is a schematic view showing a change of Id in a sensor of an embodiment (solid line) and a change of Id in a reference element (broken line);

FIG. 36 is a schematic cross-sectional view of a structure provided with a carrier control layer in a sensor using a graphene film of an embodiment;

FIG. 37A is a Id-Vg characteristics diagram before and after measurement in a sensor illustrated in FIG. 36, FIG. 37B is a Id-Vg characteristics diagram due to a difference in concentration of an object to be detected;

FIG. 38 is a schematic perspective view of a supermolecule provided on a surface of a graphene film of a sensor of an embodiment;

FIG. 39 is a diagram illustrating a molecular structure of a supermolecule;

FIG. 40 to FIG. 44 are schematic perspective views of a supermolecule provided on a surface of a graphene film of a sensor of an embodiment; and

FIG. 45A to FIG. 46C are schematic cross-sectional views of an example of a specific use method of a sensor of an embodiment using a graphene film.

### DETAILED DESCRIPTION

According to one embodiment, a sensor includes a graphene film and at least two electrodes. The graphene film has an opening. The opening dominantly has either a zigzag edge or an armchair edge. The two electrodes electrically contact the graphene film, for reading a change in electric characteristics of the graphene film due to coaction with an object to be detected.

Embodiments will be described below with reference to drawings. Note that the same reference numerals are applied to the same elements in each drawing.

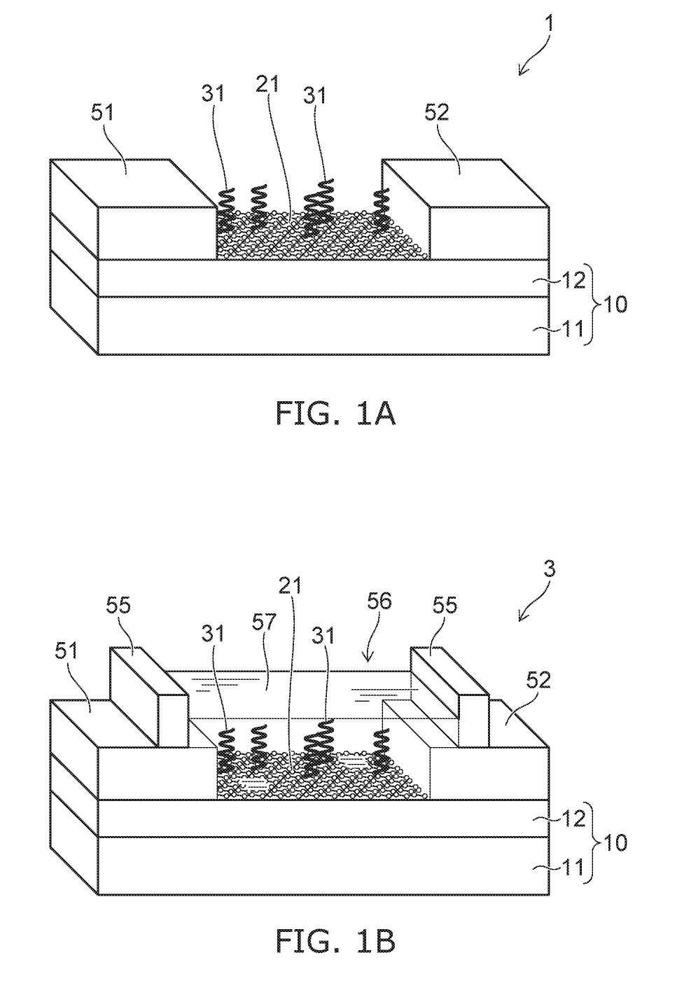

FIG. 1A is a schematic diagram of a sensor 1 of the embodiment.

The sensor 1 of the embodiment has a foundation 10, a graphene film 21 provided on the foundation 10, and at least two electrodes (first electrode 51 and second electrode 52).

The sensor 1, for example, has a field effect transistor (FET) structure. Alternatively, a wheatstone bridge circuit may be formed by the sensor 1.

The foundation 10 has a substrate 11, and a foundation film 12 provided on the substrate 11. The graphene film 21 is provided on this foundation film 12. Alternatively, the graphene film 21 may be provided on the surface of the substrate 11 without providing it on the foundation film 12. Moreover, a circuit or transistor not illustrated in the drawings may be formed on the substrate 11.

For example, silicon, silicon oxide, glass, and a polymeric material can be used as the material of the substrate 11. The foundation film 12, is an insulating film such as a silicon oxide film, for example. Furthermore, the foundation film 12 can also be given a chemical catalyst function for forming the graphene film 21.

The first electrode 51 and the second electrode 52 are provided on the foundation film 12 or the graphene film 21. The material of the first electrode 51 and the second electrode 52 is a metal material, for example. One of the first electrode 51 and the second electrode 52 functions as a drain electrode, and the other functions as a source electrode.

The graphene film 21 is provided between the first electrode 51 and the second electrode 52. The first electrode 51 and the second electrode 52 come in electrical contact with the graphene film 21. Electric current can flow between the first electrode 51 and the second electrode 52 via the graphene film 21.

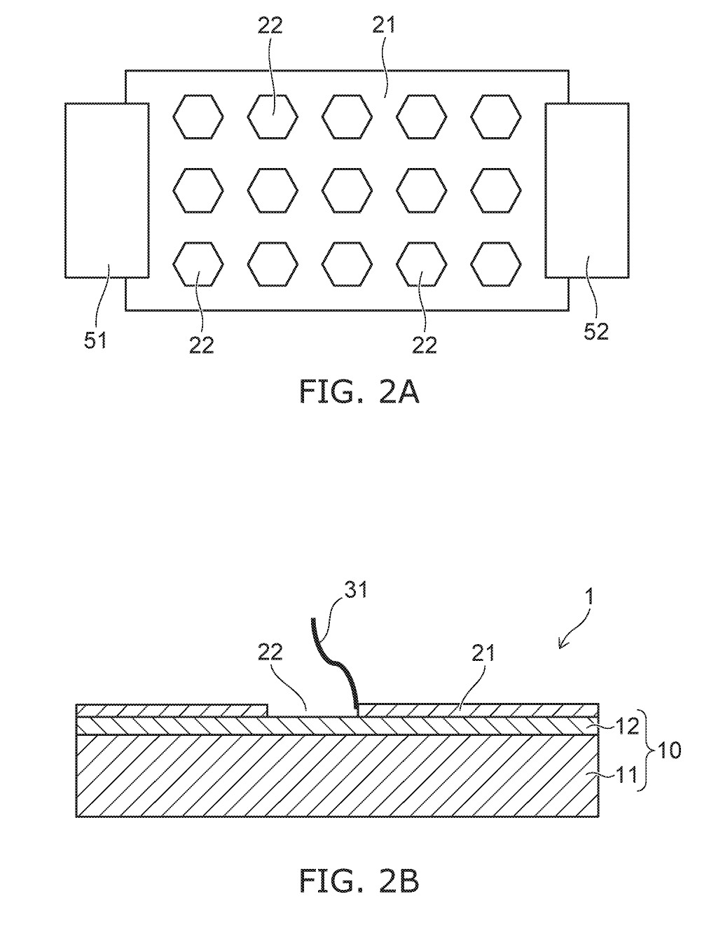

FIG. 2A is a plan view schematically illustrating an example of a planar layout of the graphene film 21 and the electrodes 51 and 52.

FIG. 2B is a schematic cross-sectional view of the sensor 1. In FIG. 2B, an illustration of the first electrode 51 and the second electrode 52 is omitted.

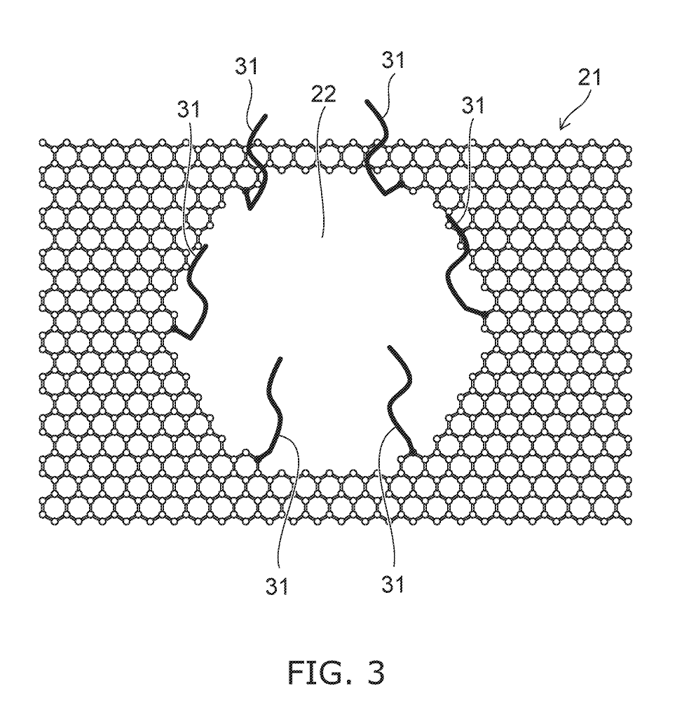

FIG. 3 is a lattice structural view of the graphene film 21. The white circles in FIG. 3 depict carbon atoms.

The graphene film 21 is configured by a honeycomb-shaped crystal lattice formed by sp² bonds of carbon atoms. The thickness of the graphene film 21 is not limited to the thickness of one carbon atom, and may be the thickness of two or more carbon atoms.

The edge of the graphene film 21 can have a zigzag edge and an armchair edge, based on the hexagonal symmetry of the graphene film 21. The zigzag edge of the graphene film 21 has a carbon skeleton similar to transpolyacetylene. The armchair edge of the graphene film 21 has a carbon skeleton similar to cis polyacetylene.

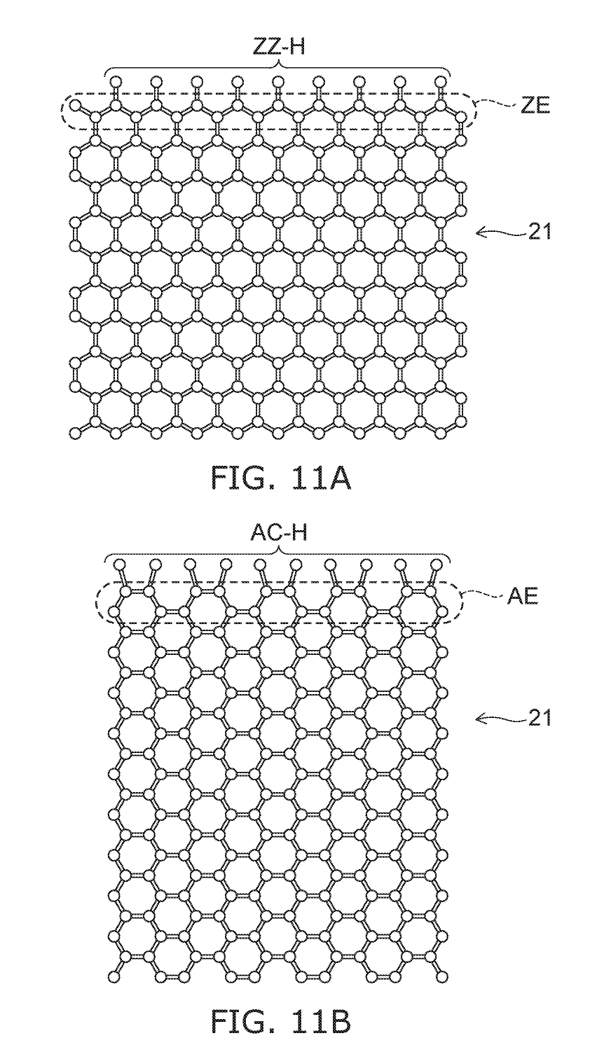

FIG. 11A is a schematic diagram for describing a zigzag edge ZE of the graphene film 21, and FIG. 11B is a schematic diagram for describing an armchair edge AE of the graphene film 21.

Hydrogen terminating the carbon of the zigzag edge ZE is illustrated as ZZ-H, and hydrogen terminating the carbon of the armchair edge AE is illustrated as AC-H.

When all carbon that can terminate at the edge of the graphene film 21 terminates by hydrogen, carbon having a hydrogen bond and carbon not having a hydrogen bond alternately align one atom at a time in an extending direction of the edge on the zigzag edge ZE. On the armchair edge AE, carbon having a hydrogen bond and carbon not having a hydrogen bond alternately align two atoms at a time in the extending direction of the edge.

In the graphene film, two individual sublattices appear due to the structural peculiarity of the honeycomb-shaped structure.

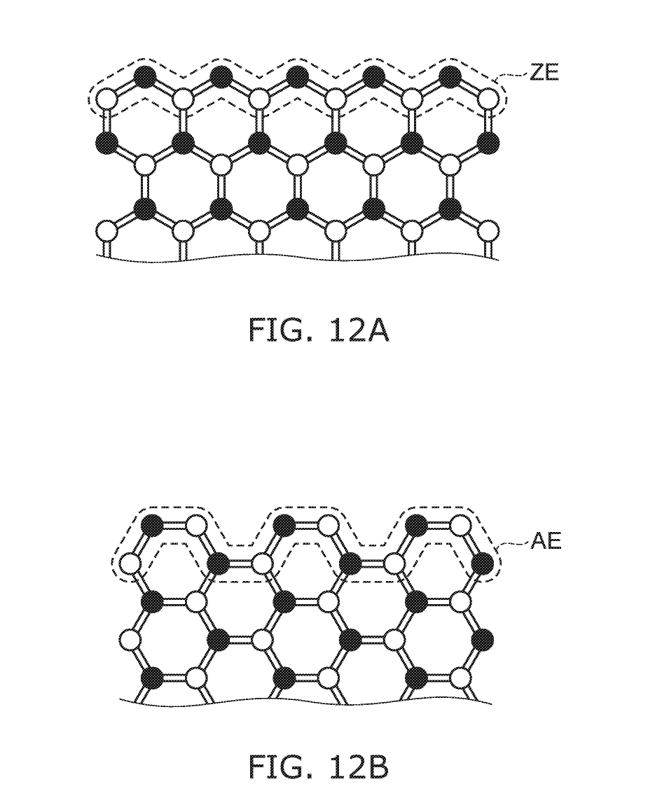

FIG. 12A is an arrangement diagram of the two sublattices on the zigzag edge ZE of the graphene film. FIG. 12B is an arrangement diagram of the two sublattices on the armchair edge AE of the graphene film. In FIGS. 12A and 12B, the black circles and white circles illustrate each individual sublattice.

On the zigzag edge ZE, only one of the two sublattices appears on the parallel line of the edge. On the armchair edge AE, the two sublattices appear alternately on the parallel line of the edge.

According to the embodiment, openings 22 are formed on the graphene film 21 as illustrated in FIG. 2A, FIG. 2B, and FIG. 3. As illustrated in FIG. 2B, the openings 22 are bottomed openings in which the foundation film 12 is the bottom. At least one opening 22 is formed on the graphene film 21 on the region between the first electrode 51 and the second electrode 52.

Either of the zigzag edge and the armchair edge dominantly appears on the edge forming the contour of the openings 22. The outline of the openings 22 is a polygon such as a hexagon. The zigzag edge or armchair edge dominantly appears on the portion corresponding to the sides of this polygon (hexagon).

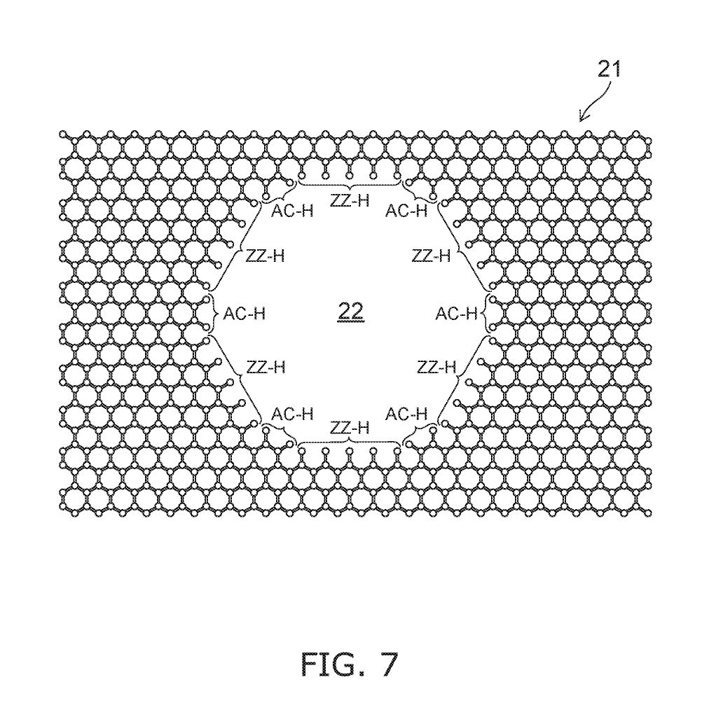

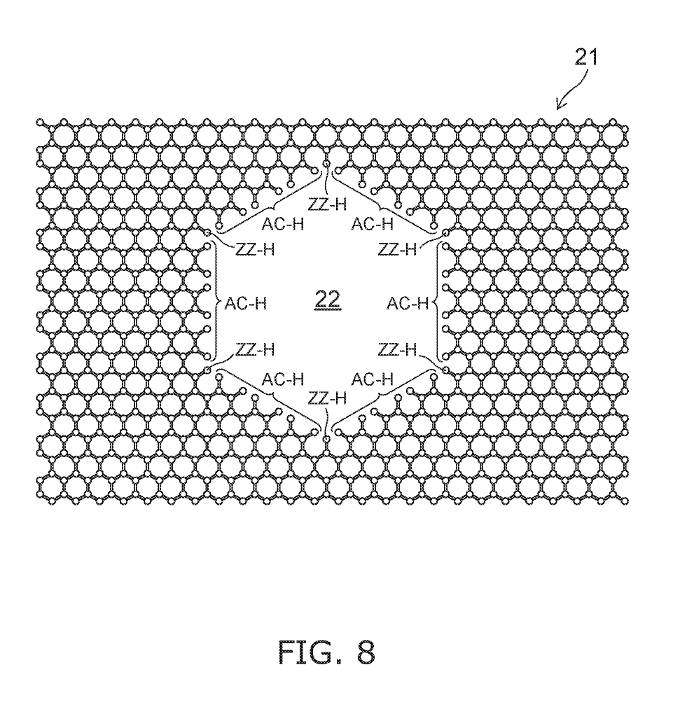

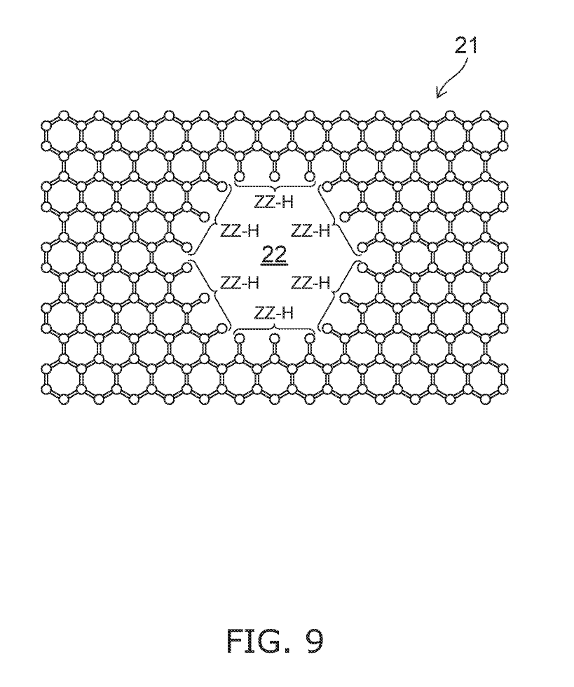

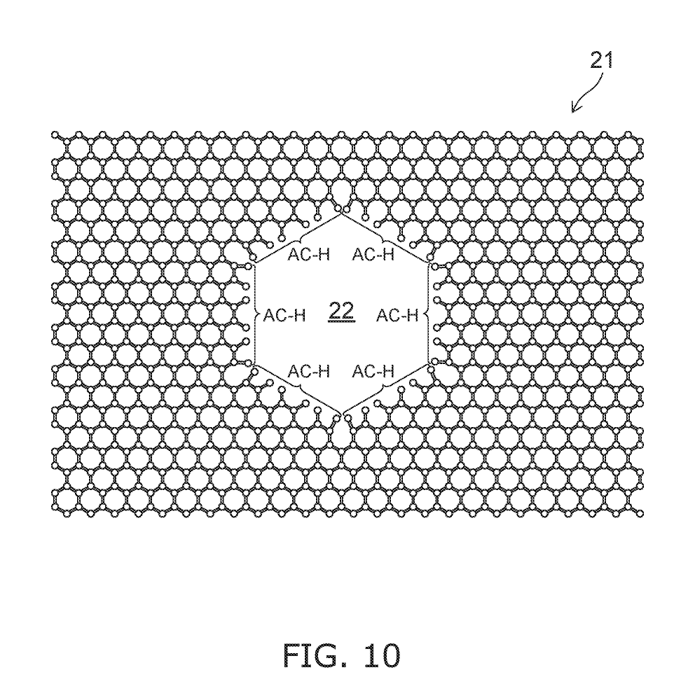

FIG. 7 and FIG. 9 illustrate examples of the openings 22 dominantly having the zigzag edge, and FIG. 8 and FIG. 10 illustrate examples of the openings 22 dominantly having the armchair edge.

With the openings 22 dominantly having the zigzag edge, the number of carbons that configure the zigzag edge is larger than the number of carbons that configure the armchair edge. Conversely, with the openings 22 dominantly having the armchair edge, the number of carbons that configure the armchair edge is larger than the number of carbons that configure the zigzag edge.

Moreover, sides in which the zigzag edge dominantly appears in polygons approximating the openings 22 (sides controlled to a zigzag edge) have a portion in which hydrogen ZZ-H having terminated nitrogen of the zigzag edge is aligned in a sequence of three or more in the extending direction of the zigzag edge.

Sides in which the armchair edge dominantly appears in polygons approximating the openings 22 (sides controlled to an armchair edge) have a portion in which hydrogen AC-H having terminated nitrogen of the armchair edge are aligned in a sequence of three or more in the extending direction of the armchair edge.

In the example illustrated in FIG. 7, the six sides of the hexagon are formed in a zigzag edge, and an armchair edge is formed between two sides (corners). The number of carbons configuring a zigzag edge (or the number of hydrogens ZZ-H terminated to a zigzag edge) is larger than the number of carbons configuring an armchair edge (or the number of hydrogens AC-H terminated to an armchair edge).

In the example illustrated in FIG. 8, the six sides of the hexagon are formed in an armchair edge, and a zigzag edge is formed between two sides (corners). The number of carbons configuring an armchair edge (or the number of hydrogens AC-H terminated to an armchair edge) is larger than the number of carbons configuring a zigzag edge (or the number of hydrogens ZZ-H terminated to a zigzag edge).

The openings 22 illustrated in FIG. 9 only have zigzag edges, and the openings 22 illustrated in FIG. 10 only have armchair edges.

Zigzag edges and armchair edges may be intermixed on a side. In a case where the number of carbons configuring a zigzag edge is larger than the number of carbons configuring an armchair edge on this side, it can be said that this is a side in which zigzag edges dominantly appear (side controlled to a zigzag edge). Inversely, in a case where the number of carbons configuring an armchair edge is larger than the number of carbons configuring an zigzag edge, it can be said that this is a side in which armchair edges dominantly appear (side controlled to an armchair edge).

As illustrated in FIG. 3, probe molecules 31 are bonded to edges of the openings 22. The probe molecules 31 are bonded to the graphene film 21 by covalent bonding. Alternatively, the probe molecules 31 are physically adsorbed to the graphene film 21 by a Van der Waals force. In this case, the probe molecules 31 are not limited to the edges of the graphene film 21, and may be adsorbed to the surface of the graphene film 21 as illustrated in FIG. 1A. Alternatively, polycyclic aromatics are formed on the terminal of the probe molecules 31, and the probe molecules 31 and the graphene film 21 are adsorbed by the affinity between polycyclic aromatics. Even in this case, the probe molecules 31 are not limited to the edges of the graphene film 21, and may be adsorbed to the surface of the graphene film 21.

The edges of the graphene film 21 are terminated by hydrogen. For example, hydrogen on the terminal of the edges of the openings 22 is replaced by terminals of the probe molecules 31, and the probe molecules 31 are covalently bonded to the carbon on the edges of the openings 22. The carbon atoms on the edges of the openings 22 maintain an sp² bonding.

The probe molecules 31 have a property for selectively recognizing specific substances (objects to detect). The probe molecule 31 includes, for example, at least one of an antibody, fragment antibody with only the antigen recognition site of the antibody removed, nucleic acid, artificial nucleic acid, aptamer, peptide aptamer, enzyme, coenzyme, fluorescent dye, and a compound containing a donor structure in an electron-transfer-type fluorescent probe represented by a photoinduced electron transfer (PeT) method, and phenylboronic acid. Examples molecules used in PeT include DMAX, hydroxyphenyl fluorescein (HPF), aminophenyl fluorescein (APF) and the like.

The graphene film 21 can have a unique electronic state near the edges. It is easy for the electronic state thereof to change due to the association, separation, or reaction of the probe molecules 31 and the object to be detected. By reading the change in electrical properties of such a graphene film 21 using the first electrode 51 and the second electrode 52, a specific object to be detected can be sensed at high sensitivity.

In the graphene film 21, the bonding energy between the terminal hydrogen and carbon of a zigzag edge is lower than the bonding energy between the sp³ bonded carbon and hydrogen, and the bonding energy between the terminal hydrogen and carbon of an armchair edge is lower than the bonding energy between the terminal hydrogen and carbon of a zigzag edge. Therefore, armchair edges have higher reactivity than zigzag edges, and the terminal hydrogen of armchair edges is easily replaced by the probe molecules 31.

FIG. 3 illustrates an example in which probe molecules 31, for example, selectively bond to the armchair edges of the openings 22 using this difference in edge reactivity.

FIG. 3 illustrates an example in which armchair edges appear on each of six edges of one opening 22 approximated to a substantial hexagon shape. In such a hexagonal opening 22, six specific edges can be made to dominantly appear on the six corners or six sides, regardless of the size of the opening 22.

For example, six probe molecules 31 can be bonded to the six armchair edges in one opening 22 using the difference in reactivity of the edges.

Here, two adjacent terminal hydrogens (AC-H in FIG. 7) each exist on the armchair edges of the six corners, but only one probe molecule 31 can be bonded to each corner portion due to, for example, an effect such as steric hindrance between probe molecules 31. Furthermore, one cycle of armchair edges is formed including a pair of two adjacent terminal hydrogens in the drawings on each corner, but only one probe molecule 31 can be bonded to the corner portions due to a steric hindrance effect between probe molecules 31 in a similar manner, even when this is formed after, for example, continuing for two or three cycles. Naturally, a molecular design can also be made wherein two probe molecules 31 are intentionally bonded to each corner portion.

In this manner, the number or density of the probe molecules 31 can be controlled by controlling the number or density of reaction sites appearing on one opening 22 (armchair edges or zigzag edges). Additionally, in a case where the number or density of openings 22 formed on the graphene film 21 are controlled, the number or density of probe molecules 31 can be controlled, sensor characteristics between devices can be controlled in any manner, and sensor characteristics can be made uniform between devices.

The openings 22 can be formed by, for example, plasma etching using a gas including hydrogen (H₂). For example, it becomes easier to form substantially hexagonal openings 22 in a stable shape by controlling the etching conditions so that etching action by hydrogen radicals is dominant.

The shape of the openings 22 is not limited to a hexagon. Moreover, openings only formed by armchair edges, or openings only formed by zigzag edges can also be formed.

In the method for making edges appear by forming the openings 22 on the graphene film 21, a desired edge can be formed by process conditions in etching processing without being affected by crystal orientation of the graphene film 21 or the like. This is a characteristic that is difficult to achieve in the outermost peripheral end of the graphene film 21.

A zigzag and an armchair are mixed on the edge structure of the outermost peripheral end due to the slope of the graphene crystal orientation, so the edge structure cannot be controlled unless the crystal orientation is strictly controlled.

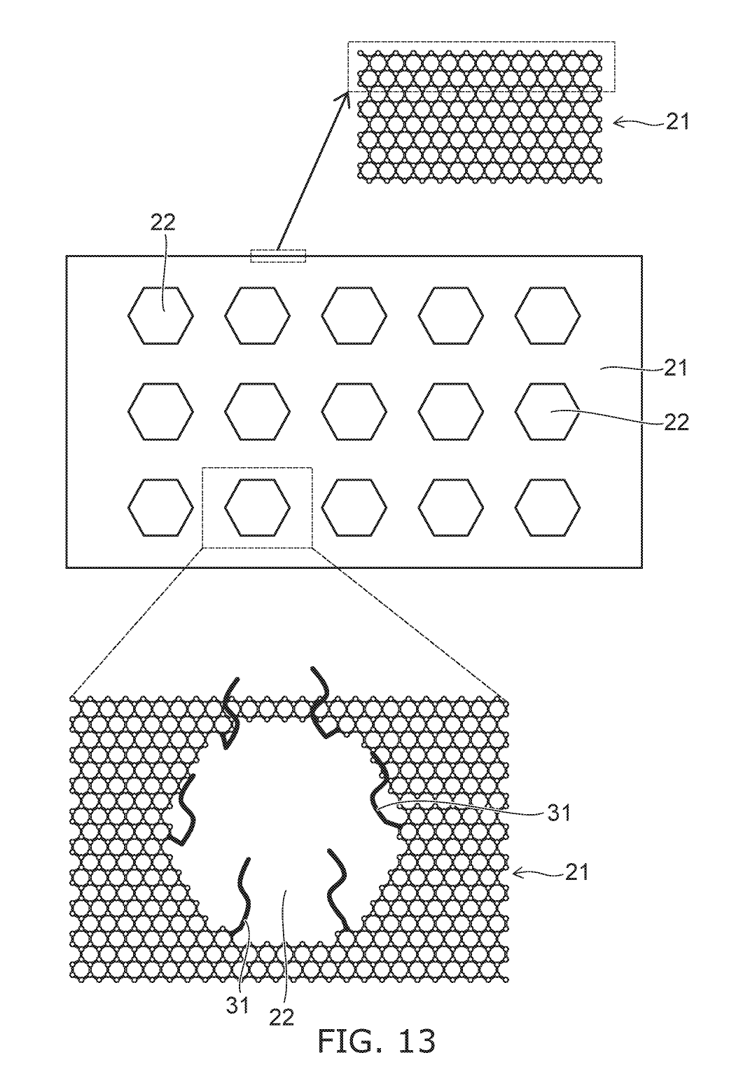

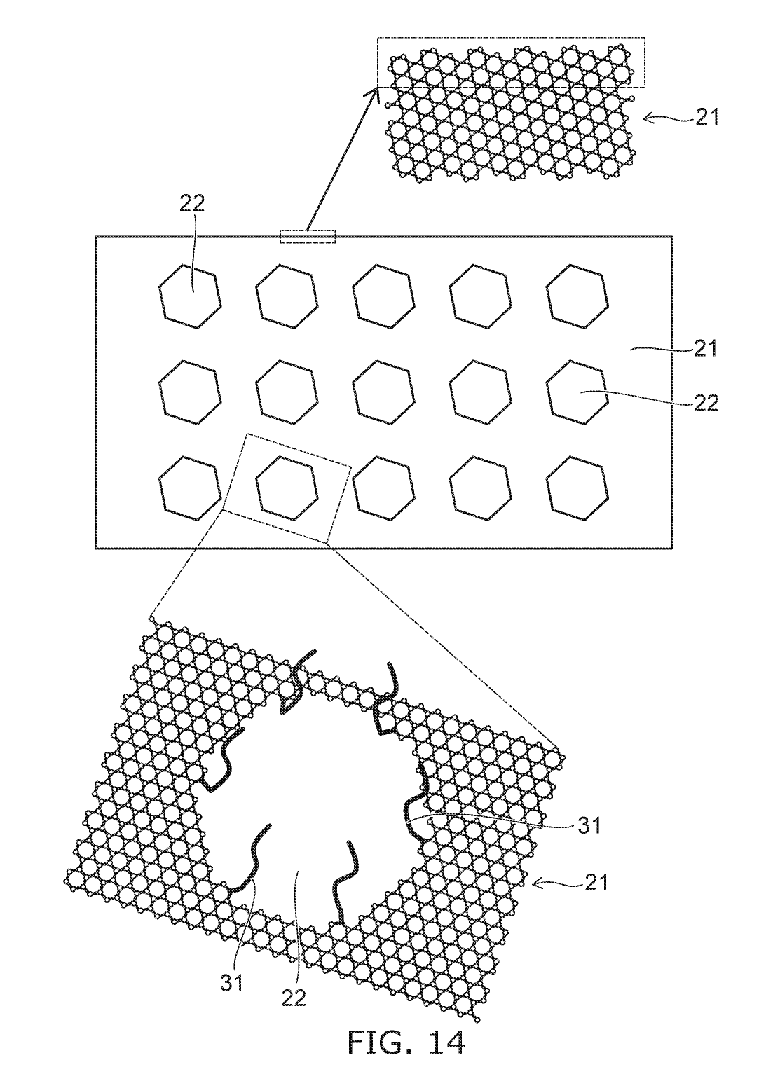

For example, when the crystal orientation of the graphene film 21 is not on a slant at all as in FIG. 13, the edge shape of the outermost peripheral end is a zigzag shape, but in actuality, it is on a slant as in FIG. 14 because it is difficult to control the crystal orientation. In this case, because the outermost peripheral end does not become parallel with the straight line of the zigzag edge or armchair edge, the outermost peripheral end has both of these mixed.

Here, the edge shape of the outermost peripheral end in FIG. 14 cannot be controlled, but according to the embodiment, a large number of edges of the openings 22 having their edges controlled exist in the graphene film 21, so the effects of the edge of the outermost peripheral end can be made substantially smaller. In this manner, there is the characteristic of it being easier to form a graphene film having a desired edge conductive effect (edge shape effect).

Different types of functional molecules can be adsorbed to the edges of the openings 22 by controlling the number or density thereof using the difference in reactivity of the edges described above.

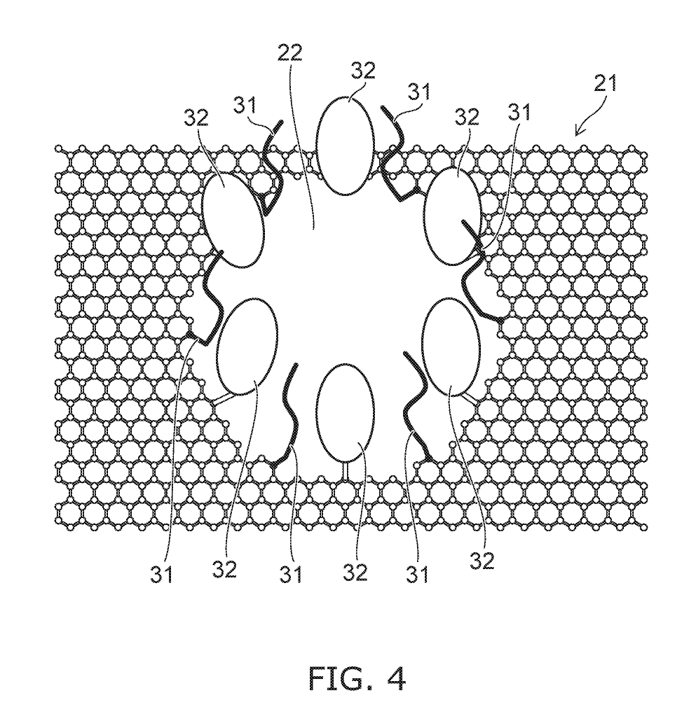

FIG. 4 illustrates an example in which, for example, probe molecules 31 are bonded to armchair edges of the openings 22, and molecules (blocking agent) 32 that block non-specific adsorption are bonded to zigzag edges.

The block molecules 32 block types of substances (molecules, ions, viruses, microorganisms or the like) different from the target substance (molecules, ions, viruses, microorganisms or the like) to which the probe molecules 31 aggregate from adhering to the graphene edge. Such a structure can reduce noise (detect a non-detection object) and detect a specific object to be detected with high sensitivity. The probe molecules 31 may be bonded to the zigzag edges, and the block molecules 32 may be bonded to the armchair edges.

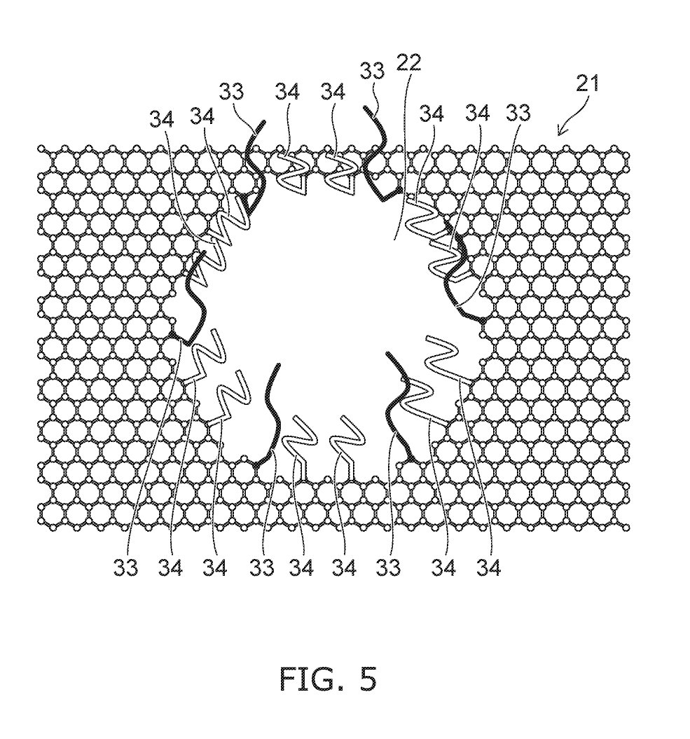

FIG. 5 illustrates an example in which first probe molecules 33 are bonded to armchair edges, and second probe molecules 34, which are a different type from the first probe molecules 33, are bonded to zigzag edges.

Here, for example, the first probe molecules 33 can be made to assemble with a reaction product due to the enzyme using an enzyme in the second probe molecules 34. In this case, detection can be carried out even with a substance that is difficult to recognize, before reacting with the enzyme.

Alternatively, when the second probe molecules 34 have a structure of a substance, for example, that bonds with a specific receptor of an immune cell (for example, ligand), the immune cell recognizes the bonding of ligand to the receptor, and emits an endocrine substance such as cytokine. Here, in a case where the first probe molecules 33 are made to aggregate with this emitted endocrine substance, it can be detected whether an immune cell in which a specific receptor is realized exists.

Furthermore, by controlling the size of the openings 22 and the size of the probe molecules, the number or density of probe molecules in one opening 22 can be controlled without using the difference in reactivity of the edges of the openings 22.

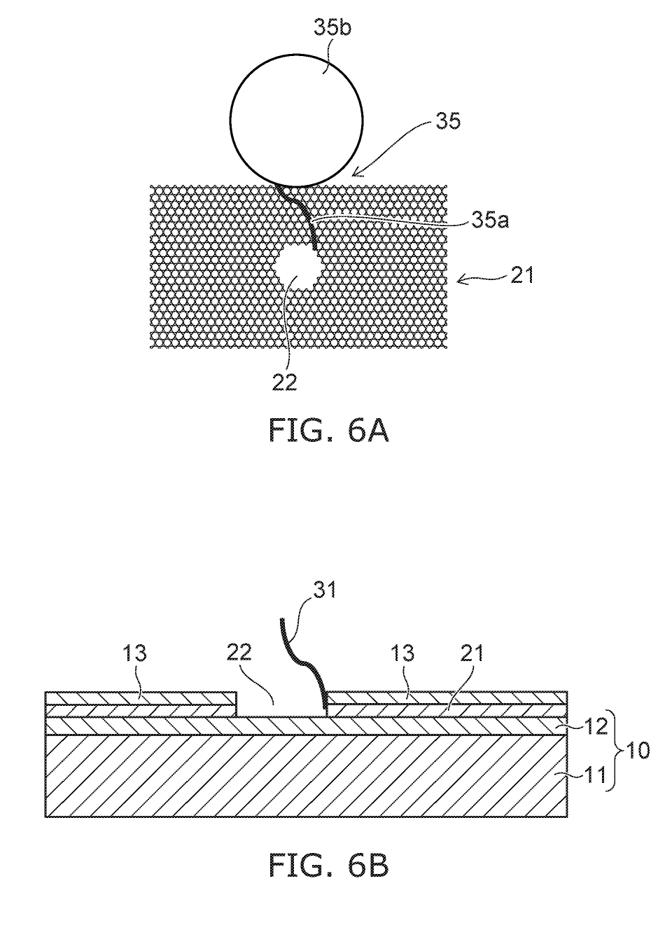

For example, because a plurality of probe molecules cannot bond with one opening 22 due to steric hindrance between probe molecules in a case where the probe molecules are made larger than the size of the openings 22, only one probe molecule 35 can bond with one opening 22 as illustrated in FIG. 6A, or no more than two probe molecules 35 can bond, regardless of the number of formation sites (armchair edges or zigzag edges) of the probe molecules.

The probe molecule 35 illustrated in FIG. 6A has an anchor part 35a bonded to an edge of the opening 22, and a head part 35b that aggregates or reacts with the object to be detected.

FIG. 6B is a schematic cross-sectional view of an example in which a protective film (surface coat film) 13 is formed on the surface of the graphene film 21.

The protective film 13 covers the surface of the graphene film 21, not including the opening 22. Such a protective film 13 prevents adsorption or approximation of an object to be detected to parts other than the probe molecules 31 on the graphene film 21. Furthermore, the protective film 13 prevents electric characteristics of the graphene film 21 from changing due to contaminants such as molecules, ions, viruses, microorganisms, or the like adsorbing or approximating a sensing atmosphere on the surface of the graphene film 21. Additionally, the protective film 13 prevents peeling of the graphene film 21 and realizes a reduction of measurement noise.

The protective film 13 is, for example, an insulating film such as silicon oxide film or silicon nitride film, a layered compound film such as boron nitride (BN), or an organic film that suppresses adsorption of protein and the like.

According to the embodiment, the probe molecules 31 can be bonded to side walls of the openings 22 even in a case where the surface of the graphene film 21 is coated.

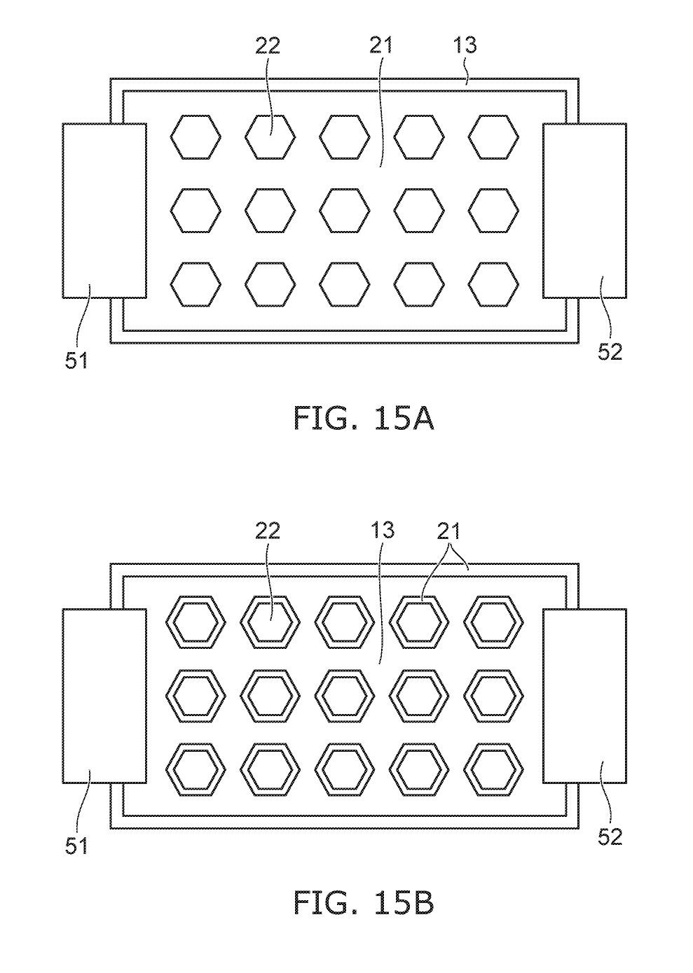

FIG. 15A illustrates an example in which the protective film 13 is formed on the end of the graphene film 21. The edges of the end of the graphene film 21 are covered by the protective film 13. The protective film 13 prevents bonding of the probe molecules to the end of the graphene film 21. Probe molecules can be bonded on the edges of the openings 22 because the edges are not covered by the protective film 13. The number of probe molecules can be controlled by the size and number of openings 22.

In the example illustrated in FIG. 15B, the protective film 13 is formed on the surface of the graphene film 21 not including near the openings 22 and the end of the graphene film 21. The carbon atoms near the openings 22 are not covered by the protective film 13, and are exposed.

The protective film 13 prevents non-selective adsorption or residue of the probe molecules or objects to be detected on the surface of the graphene film 21. Furthermore, the thickness of the protective film 13 may be, for example, 10 nm or greater to prevent change in the characteristics of the graphene film 21 due to proximity of an object to be detected.

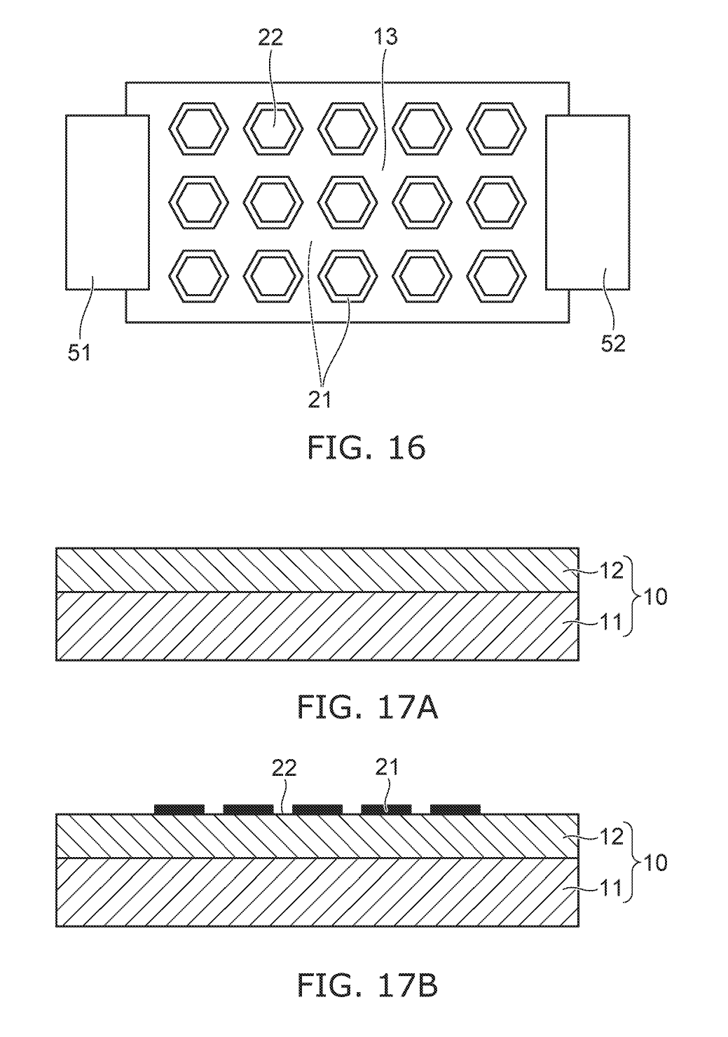

As illustrated in FIG. 16, the surface and end of the graphene film 21 may be covered by the protective film 13, not including near the openings 22.

Because the electronic state near the edges of the graphene film 21 is sensitive to changes in the surrounding environment, the electric characteristics of the graphene film 21 can be changed, for example, due to co-action of the edges of the openings 22 and the object to be detected, even in a case where probe molecules are not formed on the graphene film 21.

When appropriate probe molecules are used with the object to be detected, a higher sensitivity can be obtained for a specific object to be detected.

Alternatively, probe molecules of a different type than the probe molecules bonded to the edges of the openings 22 may be adsorbed to the surface of the graphene film 21. An example being that probe molecules, having, for example, pyrenyl groups on the terminal, to be adsorbed to the surface of the graphene film 21 by the Van der Waals force, may be adsorbed to the surface of the graphene film 21.

Furthermore, the probe molecules 31 can be bonded to edges other than the edges of the openings 22 on the graphene film 21.

Next, the manufacturing method of the sensor 1 of the embodiment illustrated in FIG. 1A will be described.

As illustrated in FIG. 17A, for example, an insulating film (foundation film) 12 for preventing discharge is formed on the substrate 11 of n⁺ type silicon. The formation of the insulating film 12 may be omitted when the substrate 11 is an insulator.

The graphene film 21 is formed on this insulating film (foundation film) 12. For example, the graphene film 21 is formed by a transfer method from graphite, chemical vapor deposition (CVD) method, a bottom-up growth method, or the like. With a transfer method, a graphene film 21 in which an opening pattern is formed by printing technology or the like may be adhered to the foundation film 12.

Openings 22 are formed on the graphene film 21 as illustrated in FIG. 17B, for example, by plasma etching using a gas including argon and hydrogen as described above.

Alternatively, the foundation film 12 may be patterned in advance, and a graphene film 21 having openings 22 may be formed on this patterned foundation film 12 by, for example, a CVD method or the like. Alternatively, a graphene film 21 having openings 22 can be formed using, for example, a bottom-up method represented by polymer synthesis.

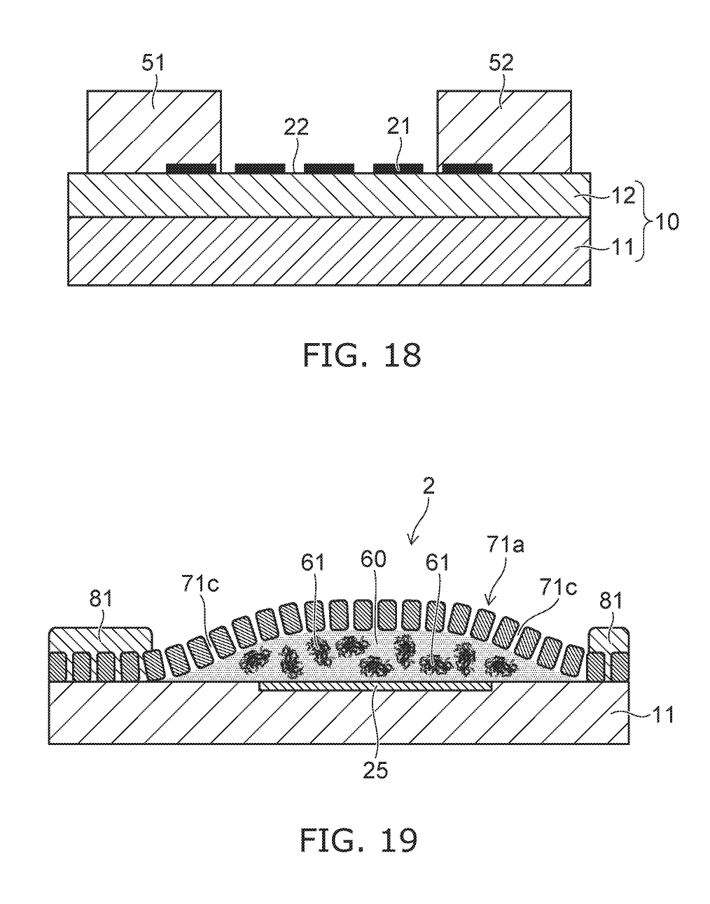

After forming the graphene film 21, the first electrode 51 and the second electrode 52 are formed as illustrated in FIG. 18.

After this, a well 56 for storing a fluid 57 may be formed on the graphene film 21 as in sensor 3 illustrated in FIG. 1B depending on the intended use of the sensor. The well 56 can be made by, for example, forming a side wall 55 of insulating film to surround the graphene film 21. The formation of the side wall 55 may be pattern processed by lithography, or may be adhered.

Furthermore, a flow path may be formed instead of a well. After forming a flow path internal structure by a sacrificing layer, the flow path can be formed by forming an insulating film around the sacrificing layer, and removing the sacrificing layer. Alternatively, it may be formed by adhering pre-formed flow path parts on another substrate.

The probe molecules 31 are bonded to the edges of the openings 22 at any time after forming the openings 22 on the graphene film 21.

The outline of the openings 22 can be made into a polygon such as a hexagon due to the etching conditions for forming the openings 22 on the graphene film 21 or the like, and the sides of this polygon can be controlled to either a zigzag edge or an armchair edge. The corners of a polygon in which the sides are controlled to a zigzag edge or an armchair edge have a different electronic state density from the sides.

FIG. 19 is a schematic cross-sectional view of the sensor 2 of another embodiment.

The sensor 2 has a sensor element 25, a liquid material 60 provided on the sensor element 25, and a thin film 71a.

The sensor element 25 electrically detects change in surface charge (electronic state). For example, the sensor element 25 is an FET using the graphene film 21 in the embodiment described above. Alternatively, the sensor element 25 is an ion-sensitive (IS)-FET having a sensitive film formed on a semiconductor FET.

The liquid material 60 contacts the surface of the sensor element 25, and is provided in, for example, a dome shape. The liquid material 60 includes probe molecules having a property for selectively recognizing a specific substance, and water.

The thin film 71a covers the liquid material 60. A plurality of through-holes 71c is formed on the thin film 71a. One end of the through-holes 71c (lower end) leads to the liquid material 60, and the other end (upper end) leads to the atmosphere on the upper surface side of the thin film 71a.

The diameter of the through-holes 71c is, for example, 10 nm or less, and furthermore 3 nm or less. Such a large number of microscopic through-holes 71c can be formed using, for example, phase separation of a self-organizing material.

The graphene film (or sensitive film) of the sensor element 25 is formed on the substrate (or foundation) 11. For example, a plurality of a graphene film (or sensitive film) is disposed in an array on the substrate 11. A plurality of the liquid material 60 is disposed in an array on the plurality of graphene films (or sensitive films).

The probe molecules associate to a target substance (object to be detected) in the liquid with substrate specificity. For example, an antibody, aptamer, peptide aptamer, phenylboronic acid, or the like are given as such a probe molecule. The probe molecules alternatively promote a chemical reaction by recognizing a target substance in the liquid with substrate-specificty. For example, an enzyme, coenzyme, antibody enzyme, ribozyme, or the like are given as such a probe molecule.

Here, “substrate-specificity” is a characteristic of selectively acting on a target molecule, and is a characteristic often had by the tissue-derived biomaterial described above or man-made composites thereof.

A description will be given here using enzymes as the probe molecules. Enzymes are catalytic molecules made up of protein derived from an organism, and has the characteristic of substrate-specifically recognizing a specific chemical substance, and selectively promoting a specific chemical reaction. Essentially, this is a material used when an organism decomposes or digests a substance, so it has a characteristic of realizing the catalytic action described above in the liquid.

Next, the manufacturing method of the sensor 2 illustrated in FIG. 19 will be described.

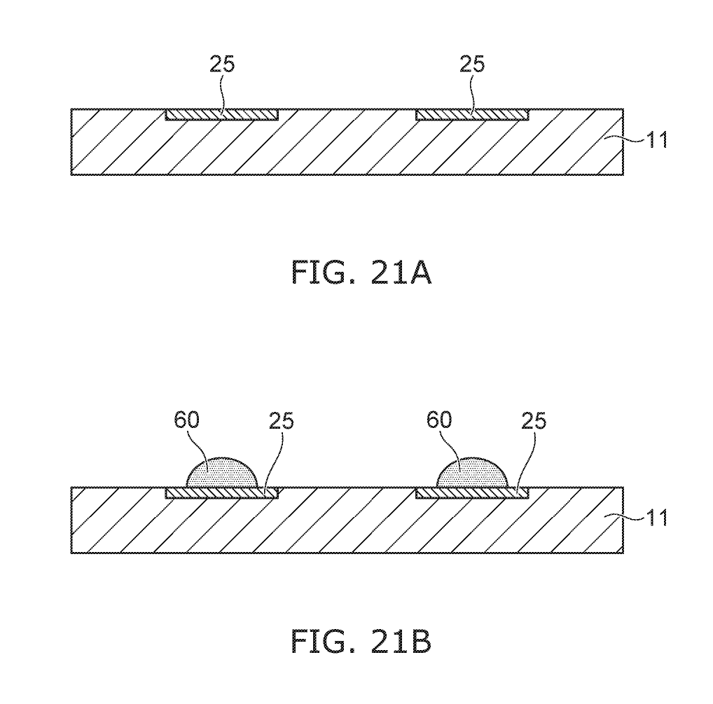

As shown in FIG. 21A, the sensor element 25 is formed on the substrate 11. Then, the liquid material 60 is formed on this sensor element 25.

For example, enzymes are taken into a high viscosity liquid including water, and the liquid material 60 is formed. Here, for example, a high viscosity liquid can be used in which a polysaccharide such as agarose has water, or in which a protein such as gelatin holds water.

Alternatively, a high viscosity liquid can be used in which a surfactant represented by the following molecular formula holds water.

Anionic surfactant: CH₃—(—CH₂—)x—CH₂—O—(—C₂H₄—O—)y—CH₂—COOH

Cationic surfactant: CH₃—(—CH₂—)x—CH₂—O—(C₂H₄—O—)y—CH₂—CO—NH—C₃H₆—NH₂

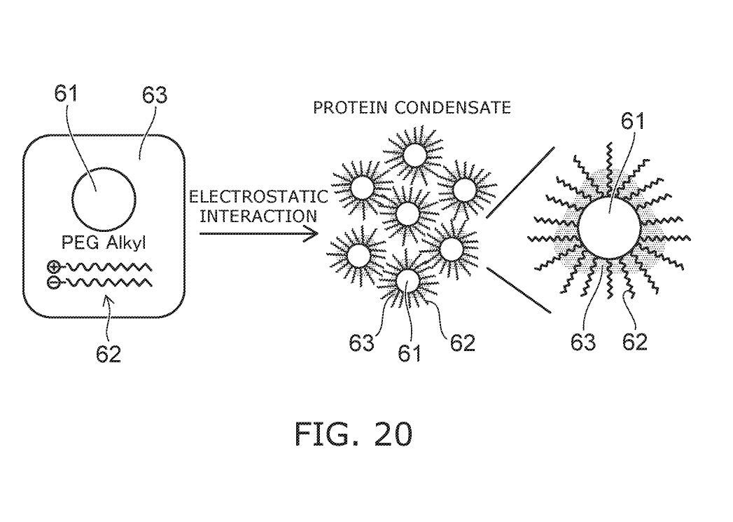

In a case where the above anionic surfactant and cationic surfactant are mixed together with enzymes and water, a carboxylic acid group of a surfactant 62 and a primary amino group surround an enzyme 61, a polyethylene glycol (PEG) chain of the surfactant 62 holds water 63, thereby forming a high concentration gel protein aggregation in which the hydrophobic alkyl chains at the front are aggregated together by electrostatic interaction as illustrated in FIG. 20.

The high viscosity liquid (liquid material) 60 obtained in this manner can be applied (supplied) on the sensor element 25 on the substrate 11 as illustrated in FIG. 21B because it has the ability to maintain the shape as a liquid drop. For example, the liquid material 60 can be locally applied to a desired position on the substrate 11 using an inkjet method, a dispenser method, or a screen printing method.

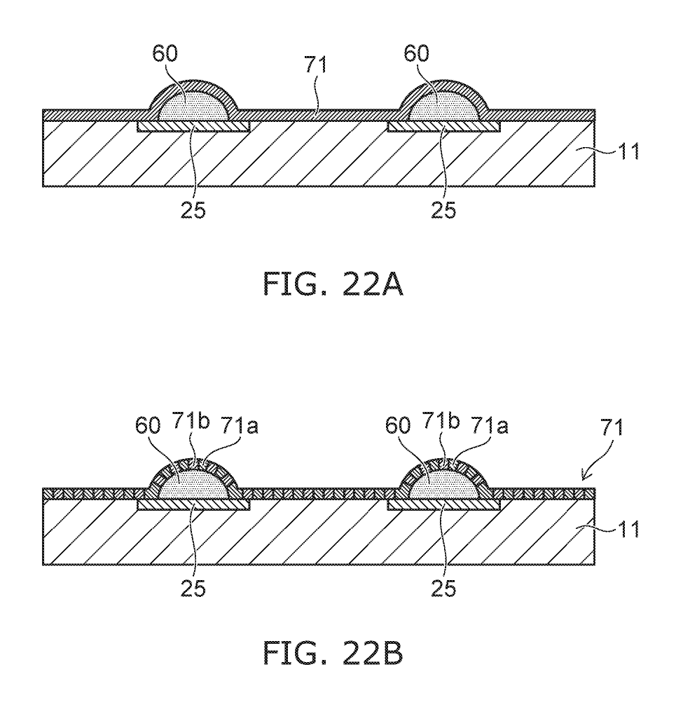

For example, a self-organizing material 71 is applied to the surface of the substrate 11 on which the high viscosity liquid material 60 is applied, as illustrated in FIG. 22A. The high viscosity liquid material 60 is not fixed that strongly to the sensor element 25. Therefore, for example, a slit coater method or the like is more preferable as a method for applying the self-organizing material 71 than a method in which centrifugal force is applied such as a spin coat method.

The self-organizing material 71 is, for example, a block copolymer in which two polymers, one hydrophilic and one hydrophobic, are bonded, the polymers being incompatible with each other. By controlling the molecular chain length ratio of these two polymers, microphase separation can be carried out on, for example, a cylinder structure including a phase 71a and a phase 71b as shown in FIG. 22B.

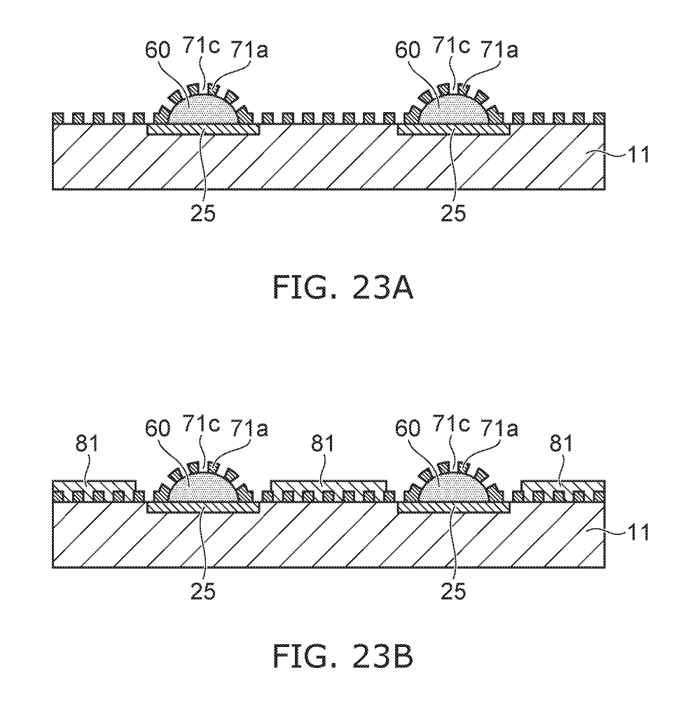

By selectively etching the one phase (for example, column of the cylinder structure) 71b, a structure can be formed having the large number of microscopic through-holes 71c made on the thin film 71a formed by the remaining phase as illustrated in FIG. 23A. In a case where the remaining phase is made to have a function as, for example, a photo-curable resin, the thin film 71a can be chemically stable.

By making the molecular weight of the polymer on the side forming the cylinder structure column of the self-organizing material 71 sufficiently small, specifically reducing it to a molecular weight of 100 or less with a repeating carbon number called an oligomer, extremely small through-holes 71c can be formed having a diameter of several nm, more specifically 3 nm or less, after the column is removed by etching.

Enzymes larger than such microscopic through-holes 71c cannot pass through the through-holes 71c. Meanwhile, the molecular size of most low molecular weight compounds called volatile organic compounds (hereby abbreviated as VOC) is smaller than the through-holes 71c, and such VOC's can easily pass through the through-holes 71c.

For example, the molecular weight of an enzyme called lysozyme is 14500 Da, and the size of such lysozyme is appropriately 4.5 nm×3.0 nm×3.0 nm. Most of the other enzymes are much larger than lysozyme.

Meanwhile, for example, formaldehyde, the hazardous substance benzene, polychlorinated biphenyl (PCB), dichlorodiphenyltrichloroethane (DDT), and morphine, cocaine, and the like, which show strong drug dependence, all of which cause sick building syndrome, are given as examples of VOC. The size of formaldehyde is approximately 0.3 nm, the size of benzene is approximately 0.6 nm, the size of PCB is approximately 1.3 nm, the size of DDT is approximately 1.2 nm, the size of morphine is approximately 0.9 nm, and the size of cocaine is approximately 1.3 nm.

Additionally, with the protein aggregation illustrated in FIG. 20 using the previously described surfactant 62, the water 63 does not leak from the through-holes 71c because it is held by the surfactant 62.

In a case where the enzyme 61 taken into the high viscosity liquid material 60 promotes a catalytic reaction with a specific VOC, when the sensor 2 is exposed to the atmosphere in which the target VOC exists, the VOC that has entered the high viscosity liquid material 60 via the through-holes 71C reacts due to the enzyme 61 in the liquid material 60, forming a reaction product in the liquid material 60.

Generally, the reaction product by the enzyme 61 is dispersed around the enzyme 61, so it is difficult to sense the generation thereof. However, because the concentration of the reaction product rises considerably in a system (liquid material 60) closed in a small space such as the embodiment, the generation of the reaction product, that is, the existence of the target VOC can be read with high sensitivity as an electric signal by the sensor element 25. The reaction product changes the electric characteristics of the graphene film (or sensitive film) of the sensor element 25.

FIG. 23B illustrates a structure in which a reinforcing film (or anchor film) 81 is formed on the thin film 71a formed on a portion where the high viscosity liquid material 60 is not applied. Such a structure raises the adhesive strength of the film 71a to the substrate 11. For example, the reinforcing film 81 can be selectively formed by a photo-resist patterning.

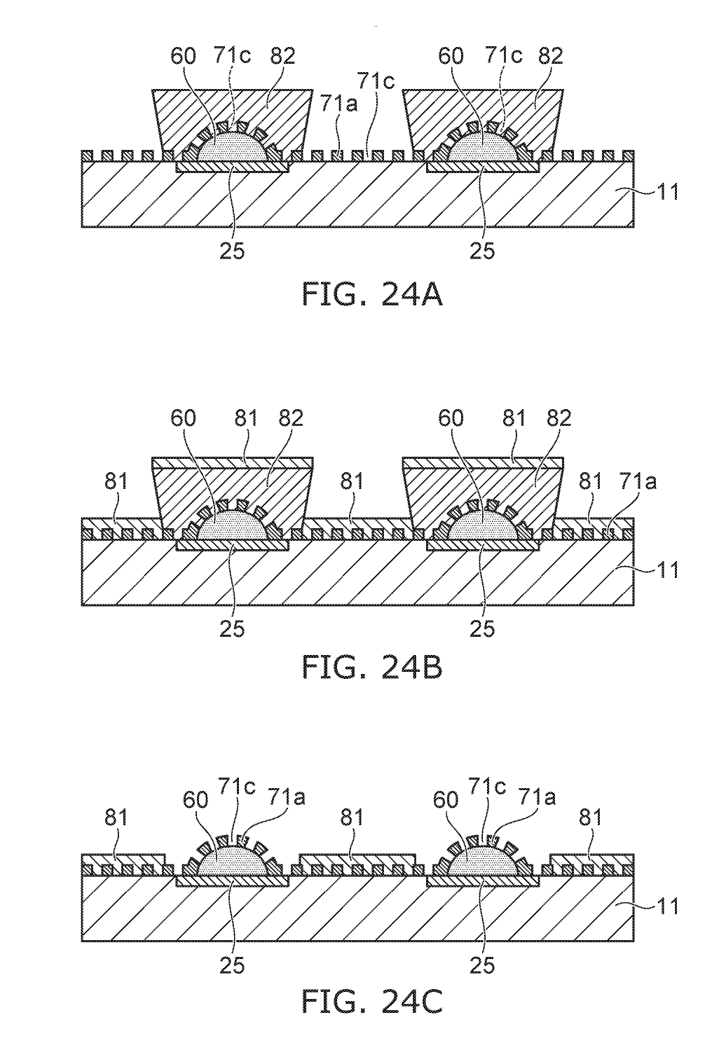

Alternatively, as illustrated in FIGS. 24A to 24C, the reinforcing film 81 can be selectively left behind using the lift off of a resistance 82.

As illustrated in FIG. 24A, the resistance 82 is formed on the portion on which the high viscosity liquid material 60 is applied. Next, as illustrated in FIG. 24B, for example, the reinforcing film 81 is formed on an upper surface of the resistance 82, and on the portion where the high viscosity liquid material 60 is not applied, using a low temperature sputtering method. Afterward, when the resistance 82 lifts off (separates), the reinforcing film 81 can be selectively left behind on the portion where the high viscosity liquid material 60 is not applied, as illustrated in FIG. 24C. The liquid material 60 on the sensor element 25 connects to the atmosphere of the object to be detected via the through-holes 71c.

A portion where the liquid material 60 including a first enzyme is applied, and a portion where the liquid material 60 including a second enzyme of a different type than the first enzyme can be formed on the same substrate 11. Such a structure can detect a plurality of types of target substances.

A plurality of types of enzymes may be taken into one high viscosity liquid material 60. Such a structure makes it possible to promote continuous chemical reactions, and a reaction product of a secondary chemical reaction can be detected even in a case where, for example, the reaction product of a primary chemical reaction is difficult to detect using the sensor element 25.

Mildew does not infiltrate into the liquid material 60 from the through-holes 71c because the through-holes 71c formed by the phase separation of the self-organizing material 71 are smaller than the size of mildew. Mildew does not occur on the sensor 2 even though it uses enzymes in a wet environment. The thickness of mycelia extending when mildew takes root on the foundation is 0.5 μm or greater, and 100 μm or less. In a case where the invention is specialized to this object, the diameter of the through-holes 71c may be 500 nm or less.

With microscopic through-holes 71c of several nm, enzymes do not enter from the exterior. Therefore, types of enzymes different from the enzyme included in the liquid material 60 do not enter into the liquid material 60. This prevents the detection of reaction products that are not the object to be detected, obtained by reaction promoting of a non-target substance. Additionally, enzymes that decompose probe molecules in the liquid material 60 are also prevented from entering.

Above, enzymes were described as the probe molecules, but the characteristic of enzymes not entering from the exterior is extremely effective when detecting a bodily fluid such as blood or the like using for example, aptamers as the probe molecules.

Aptamers having nucleic acid as the skeleton can be easily decomposed by nuclease, which is a nucleolytic enzyme existing in the body, and it was difficult until now to use aptamers for detecting bodily fluid.

According to the embodiment, because nuclease cannot pass through the microscopic through-holes 71c, aptamers can be protected from nuclease, and the sensor 2 of the embodiment can be applied to bodily fluid detection with sufficient reliability.

An aptamer can be fixed to a charge detection film (graphene film or the like) of the sensor element 25 via a linker. A target molecule (object to be detected) can be detected by electrically reading the change in a three-dimensional shape generated by an aptamer having a charged load capturing a target molecule, or the charge of the target molecule itself.

The thickness of the chain in the nucleic acid of the skeleton of the aptamer is 1.9 nm. Therefore, the free aptamer can pass through the through-holes 71c, but in a case where the aptamer is fixed on the sensor element 25 as described above, the aptamer can be prevented from breaking away from within the liquid material 60.

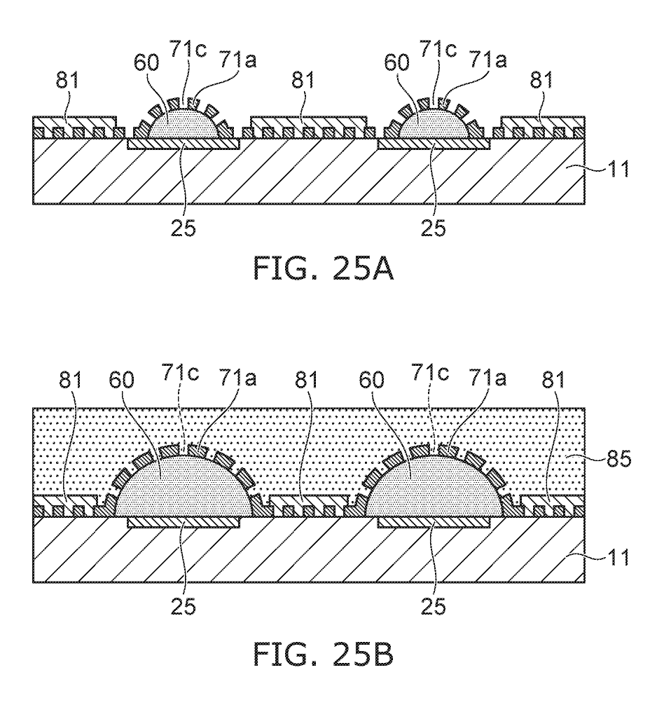

In a case where the reinforcing film (or anchor film) 81 fixing the thin film 71a is formed slightly separated from the high viscosity liquid material 60 as illustrated in FIG. 25A, the enzymes in the liquid material 60 and the VOC that has melted into the liquid material 60 can move more freely when the top of a sensor device is covered by an appropriate fluid 85 and the high viscosity liquid material 60 is diluted, as illustrated in FIG. 25B. Enzymes do not pass through the through-holes 71c even in this case.

According to the sensor 2 of the embodiment described above, a reaction product obtained by making a low molecular weight compound such as VOC react with an enzyme can be detected with high sensitivity.

Enzymes show activity in a liquid. While using the liquid material 60 including such an enzyme, the surface of the sensor device can be maintained in a dry state, and, for example, the mildew can be prevented from generating. Additionally, when the liquid material 60 has the characteristic of adsorbing water, the drying of enzymes can be suppressed.

Furthermore, because it can be fixed to a charge detection film (graphene film or the like) without chemically bonding an enzyme, there is no conformational change of the enzyme by chemical bonding. This can prevent negative effects to enzyme activity (catalytic action).

Furthermore, because other probe molecules do not enter into the liquid material 60 from the exterior via the through-holes 71c, the sensor element 25 does not detect unintended bonding or reactions. Moreover, probe molecules taken into the liquid material 60 are not decomposed because other types of enzymes do not enter from the exterior.

A probe molecule can also be used combining a coenzyme with an enzyme. Moreover, the probe molecules may be antibody enzymes or ribozymes. The molecule size of antibody enzymes is a little over 10 nm. When such antibody enzymes, and enzymes having a very large molecule size are used as probe molecules, the diameter of the through-holes 71c may be 10 nm or less.

Antibodies, aptamers, and peptide aptamers can also be used as the probe molecules. These probe molecules do not promote a target chemical reaction, but read the charge of the target bonded to the probe molecules using a sensor element. Alternatively, the sensor element 25 reads that there is conformational change of probe molecules having charge by bonding to the target.

An electric bilayer having a thickness of several nm can be formed on the liquid phase directly above the sensor element (charge detection sensor) 25. Charge transfer at a region farther from the sensor element 25 than this electric bilayer is shielded by the electric bilayer, and it may be more difficult to read using the sensor element 25.

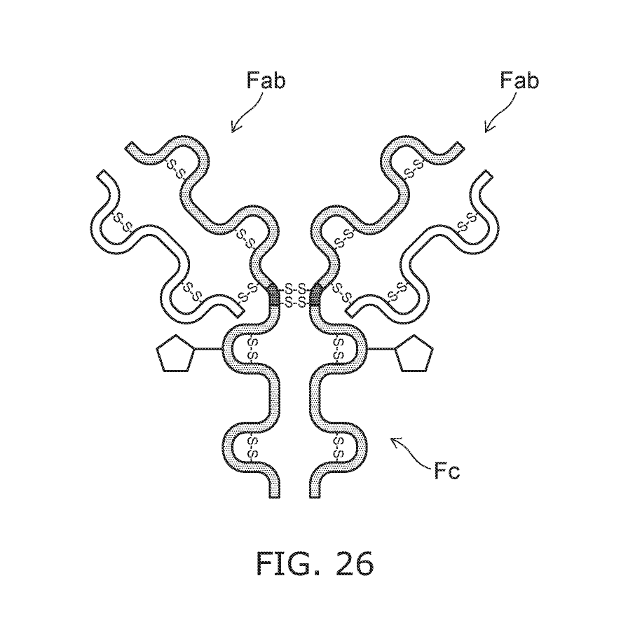

FIG. 26 is a schematic view of an IgG antibody as an example of an antibody.

Aptamers or peptide aptamers are sufficiently small in a case where they have an appropriate design, but antibodies have a size of appropriately 10 nm or greater and 20 nm or less even with the smallest IgG antibody. Therefore, the target molecule capture portion (antigen joining site) Fab is on the outer side of the electric bilayer, and the charge thereof cannot be detected.

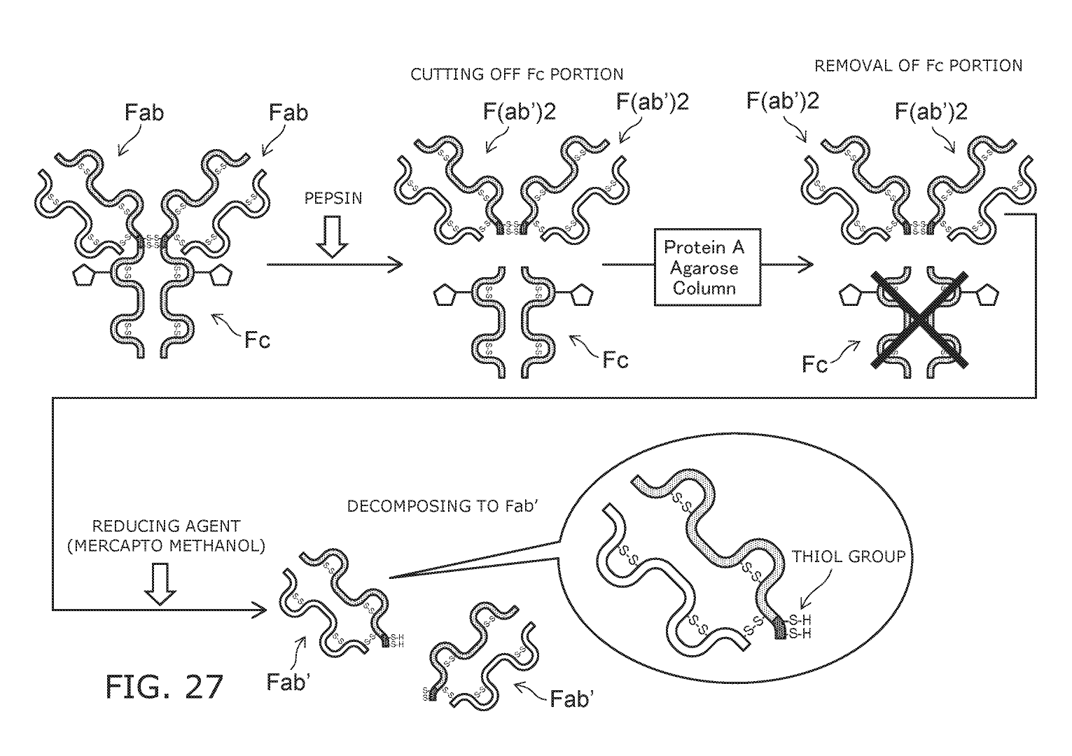

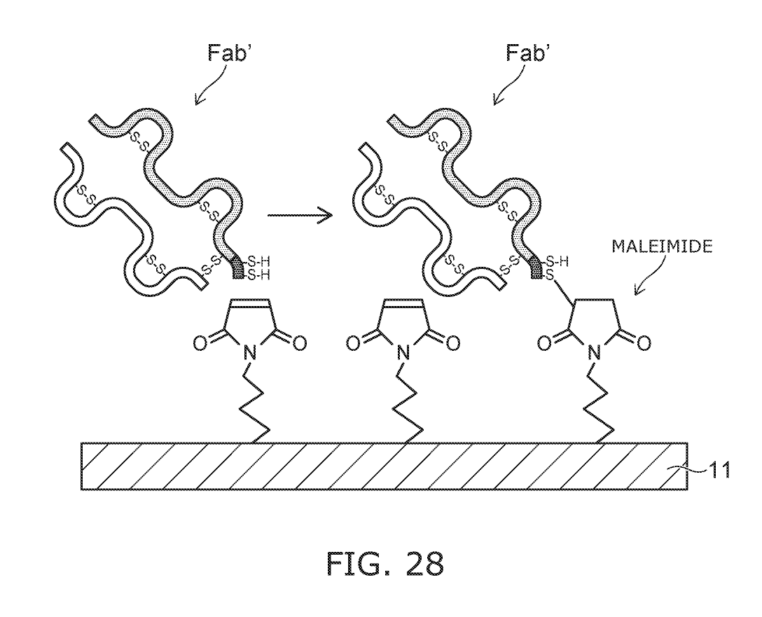

The target molecular recognition moiety Fab′ on the tip end of the IgG antibody is then cut off as illustrated in FIG. 27, and by fixing it to the foundation (substrate) layer 11 as illustrated in FIG. 28, target molecules can be captured in a region closer to the sensor element than the electric bilayer.

As illustrated in FIG. 27, for example, the Fc portion of the IgG antibody can be cut off by pepsin, the Fc portion can be removed using agarose and decomposed to the target molecular recognition moiety Fab′ using mercapto methanol. Then, as illustrated in FIG. 28, the target molecular recognition moiety Fab′ can be fixed to the foundation (substrate) 11 via maleimide. The thiol group of the target molecular recognition moiety Fab′ bonds with maleimide.

Enzymes acting in the liquid of the embodiment can be handled on a dry surface, and the characteristic of mildew not growing can be used as a device that removes hazardous gas.

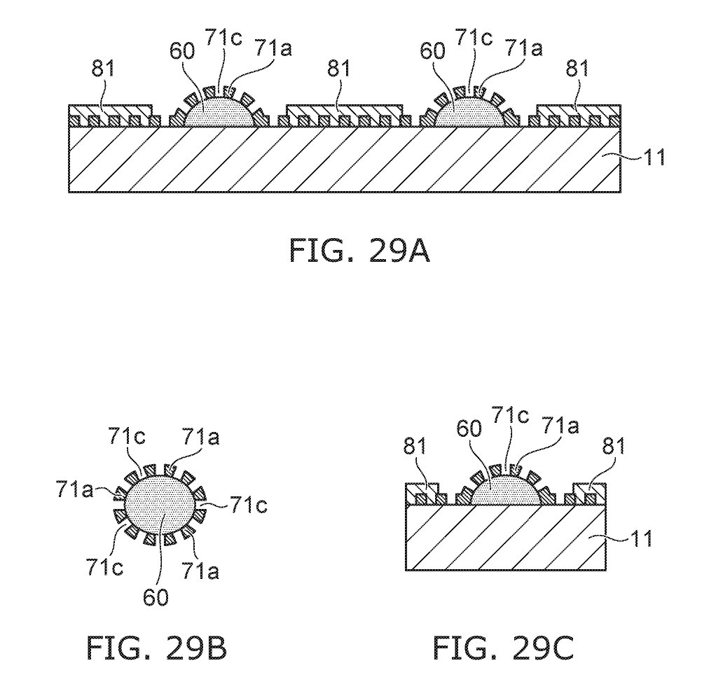

FIG. 29A is a schematic cross-sectional view of such a hazardous substance analysis device 3.

The liquid material 60 is formed on the substrate 11, and the thin film 71a is formed to cover the liquid material 60. Through-holes 71c are formed on the thin film 71a covering the liquid material 60.

Such a hazardous substance analysis device 3 may not have a sensor element because it does not necessarily need to function as a sensor. To manufacture a large area device at a low price, the foundation (substrate) 11 may be a sheet or plate supporting the liquid material 60 and thin film 71a.

For example, in a case where formaldehyde dehydrogenase, which is an aldehyde oxidase, is taken in with nicotinamide adenine dinucleotide, which is a coenzyme, formaldehyde, which is a cause of sick house syndrome, as an enzyme taken into the high viscosity liquid material 60 can be oxidized and changed into formic acid. Additionally, in a case where formic dehydrogenase is also taken in, it can be changed from formic acid to carbon dioxide.

In a case where such a device 3 is disposed as a portion of wallpaper, or disposed in any location in a room, an indoor environment with formaldehyde removed can be obtained. Furthermore, in a case where a sensor element (charge detection sensor) is installed, this device 3 itself can decompose formaldehyde, and directly measure the state in which formaldehyde residue is reduced in a room (air cleanliness). Moreover, in this case, detection can be carried out using a charge detection sensor in a formic acid state with stronger acidity than carbon dioxide without adding formic dehydrogenase.

Note that as illustrated in FIG. 29B, a sphere can be created, covering the high viscosity liquid material 60 including enzymes using the thin film 71a on which the through-holes 71c are formed, this can be included in the paint of an indoor wall, or taken in while printing wallpaper. With this application, the device shape does not necessarily need to be a sphere, and may be a hemisphere as in FIG. 29C.

According to the embodiment, the probe elements have polycyclic aromatic.

According to the embodiment, the probe molecules include, for example, at least one of an antibody, fragment antibody with only the antigen recognition site of the antibody removed, nucleic acid, artificial nucleic acid, aptamer, peptide aptamer, enzyme, coenzyme, fluorescent dye, and a compound containing a donor structure in an electron-transfer-type fluorescent probe represented by a photoinduced electron transfer (PeT) method, and phenylboronic acid.

According to the embodiment, carbon on the edges of the openings sp² bonds.

According to the embodiment, the sensor is provided with a protective film covering the surface of the graphene film, not including around the openings.

According to the embodiment, the probe molecules include at least one of an antibody, aptamer, peptide aptamer, enzyme, coenzyme, antibody enzyme, and ribozyme.

According to the embodiment, the liquid is a high viscosity liquid including at least one of a polysaccharide, protein, and surfactant.

According to the embodiment, the liquid includes aldehyde oxidase.

According to the embodiment, the diameter of the through-holes is 10 nm or less.

According to the embodiment, the diameter of the through-holes is 3 nm or less.

According to the embodiment, the sensor element is an ion sensitive (IS)-field effect transistor (FET) having a sensitive film formed on a semiconductor FET.

With the sensor 1 illustrated in FIG. 1A and the sensor 3 illustrated in FIG. 1B, a gate electrode (back gate) can be provided between the substrate 11 and the graphene film 21.

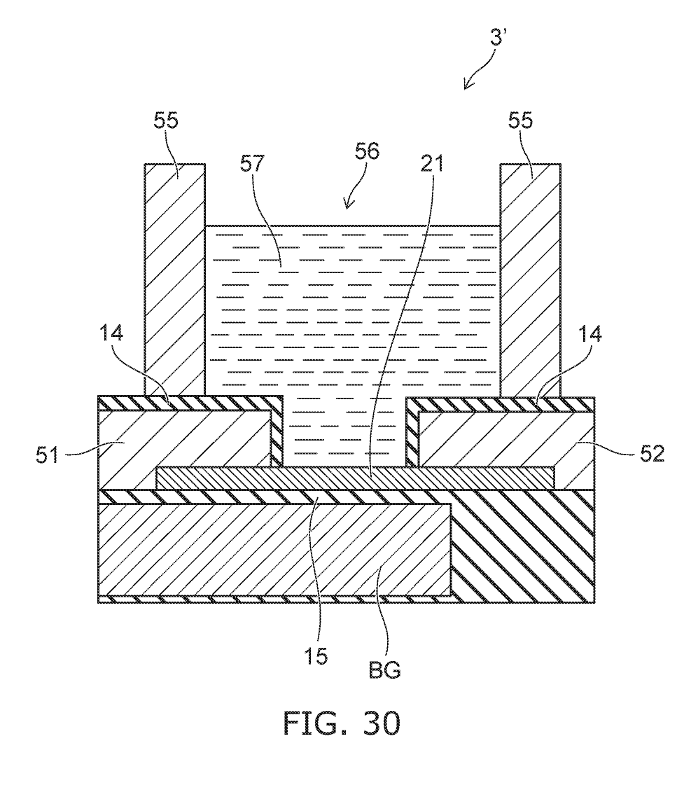

FIG. 30 is a schematic cross-sectional view of a sensor 3′ provided with, for example, a gate electrode BG in the sensor 3 shown in FIG. 1B. An illustration of the foundation is omitted.

The gate electrode BG is provided below the graphene film 21. A gate insulating film 15 is provided between the graphene film 21 and the gate electrode BG. Moreover, an insulating film 14 covers the surface of the first electrode 51 and the surface of the second electrode 52.

The sensor 3′ illustrated in FIG. 30 has an FET structure having the first electrode 51 as a source electrode, the second electrode 52 as a drain electrode, the gate electrode BG, the gate insulating film 15, and the graphene film 21 as a channel. Moreover, a reference electrode may also be provided that gives potential to a solution 57.

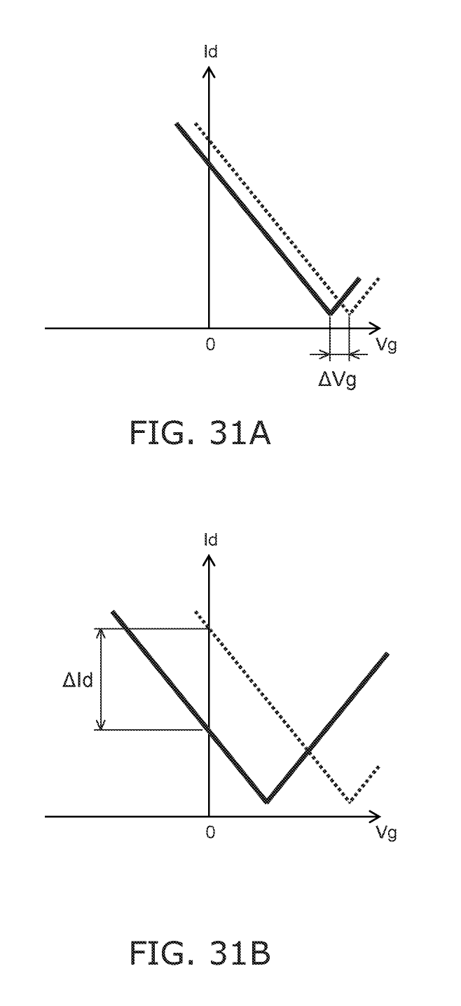

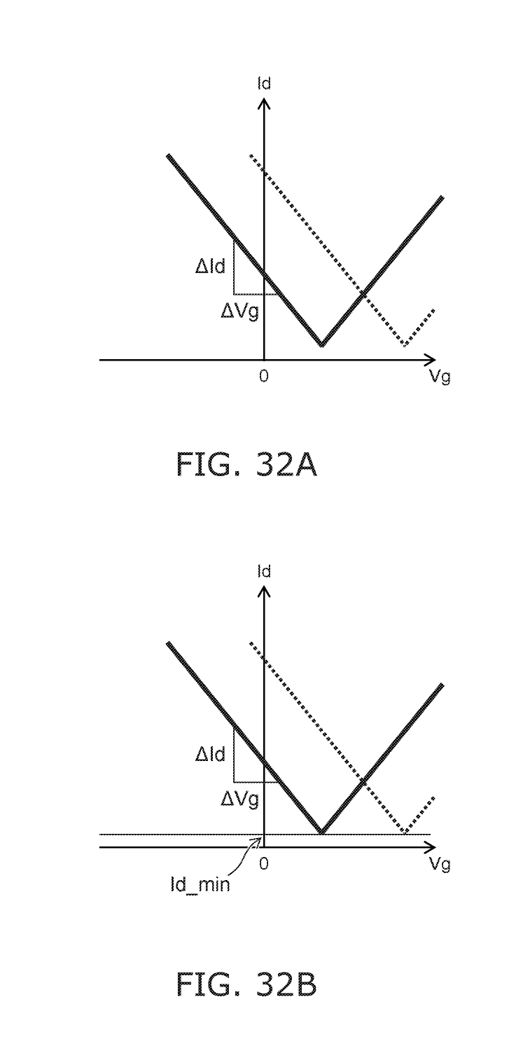

FIG. 31A to FIG. 32B are graphs illustrated an example of Id-Vg characteristics in the sensor 3′. Id indicates a current value flowing between the electrode 51 and the electrode 52 via the graphene film 21, and Vg indicates gate voltage of the gate electrode BG.

For example, before measuring the object to be detected, a constant voltage is applied between the first electrode 51 and the second electrode 52, the gate voltage Vg of the gate electrode BG fluctuates, and the current value Id is measured. Afterward, a similar operation is carried out while measuring the object to be detected. The Id-Vg characteristics before measuring the object to be detected are shown by a solid line, and the Id-Vg characteristics after measuring the object to be detected are indicated by a broken line.

The carrier amount implanted to the graphene film 21 not from the gate electrode BG, that is, the number of objects to be detected, which are the implantation sources of the carriers thereof, can be calculated from the change in Id-Vg characteristics before and after measurement. Additionally, the density or concentration of the object to be detected can be calculated by adding a bonding function to the object to be detected of the probe molecules.

For example, a change ΔVg of the gate electrode Vg in which the current value Id is at a minimum before and after measuring can be used for detection evaluation of the object to be detected as illustrated in FIG. 31A.

Alternatively, a change ΔId of Id when Vg=0 before and after measuring can be used for detection evaluation of the object to be detected as shown in FIG. 31B. In this case, hydrolysis of the fluid 57 or element destruction by the gate current Vg can be prevented. Furthermore, the measurement time can be reduced, and power consumption can be suppressed.

Alternatively, as in the example illustrated in FIG. 32A, Vg dependency of Id can be acquired in advance. Before measuring, Id when Vg=0 and Id at two points of Vg near Vg=0 are measured, and the obtained ΔId/ΔVg is stored. When measuring the object to be detected, the Id-Vg characteristics after measurement can be acquired from Id when Vg=0, and the stored data described above before measurement. When measuring, only Id when Vg=0 is measured, so element destruction is prevented, measurement time can be reduced, and power consumption can be suppressed.

In the example illustrated in FIG. 32B, the minimum value Id_min of Id, and Δid/ΔVg is stored before measurement. When measuring the object to be detected, only the gate current Vg when Id_min flow is measured, so the measurement time can be reduced, and power consumption can be suppressed.

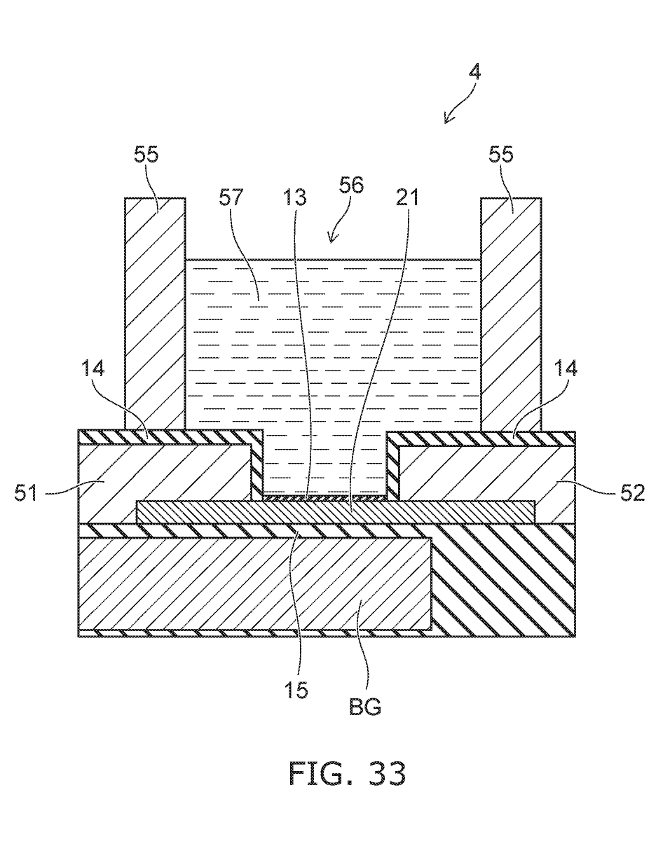

A sensor element such as a temperature sensor or a pH sensor can be consolidated on the foundation with the sensor in the embodiment described above, with the purpose of removing noise due to the implantation of carriers from other than the object to be detected. Moreover, a reference element 4 such as that illustrated in FIG. 33 can be consolidated on the foundation with the sensor described above shown in FIG. 30. The effects of the external environment (temperature, humidity, pH, liquid layer polarity, and the like) can be eliminated using another such sensor element.

With the reference element 4 in FIG. 33, probe molecules are not bonded to the graphene film 21, and the surface of the graphene film 21 in the well 56 is covered by the protective film 13.

Moreover, a region in which probe molecules are bonded to the graphene film 21 (object to be detected capture region) and a region in which the graphene film 21 is covered by the protective film 13 (reference region) can be mixed in the same well 56.

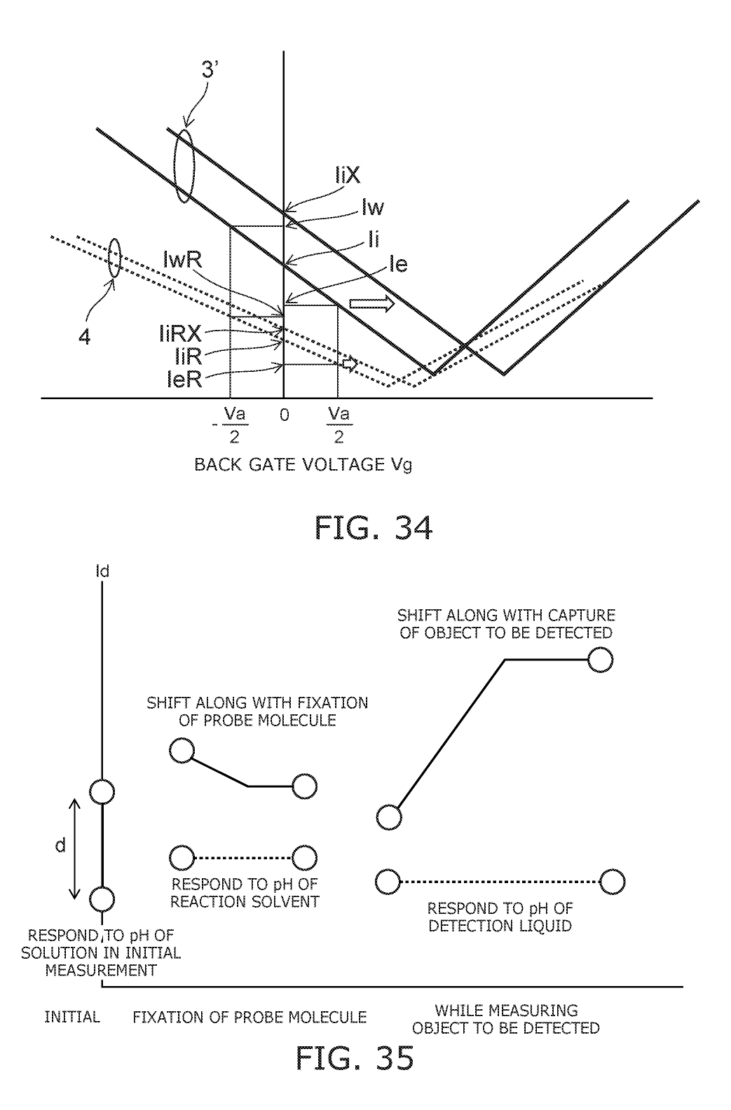

FIG. 34 is a diagram illustrated the Id-Vg shift before and after measuring the object to be detected in each of the sensor 3′ and the reference element 4.

The Id-Vg shift of the sensor 3′ that accompanies the capture of the object to be detected and the pH fluctuation can be evaluated by (Iix-Ii)/(Iw-Ie)xVa.

The Id-Vg shift of the reference element 4 that accompanies the pH fluctuation can be evaluated by (IiRX-IiR)/(IwR-IeR)xVa.

The Id-Vg shift that accompanies the capture of the object to be detected having the effects of pH fluctuation removed, can be evaluated by (Iix-Ii)/(Iw-Ie)-(IiRx-IiR)/IwR-IeR).

FIG. 35 is a schematic view showing the change of Id in the sensor 3′ (solid line) and the change of Id in the reference element 4 (broken line). The horizontal axis is the time axis.

In the initial state, a difference d due to deviation in the elements themselves is between the sensor 3′ and the reference element 4.

When a reaction solvent is supplied to the well 56 to fix the probe molecules on the graphene film 21, Id of each of the sensor 3′ and the reference element 4 shifts from the initial state in reaction to the pH of the reaction solvent thereof. Additionally, in the sensor 3′ having probe molecules fixed, Id shifts along with the fixing of the probe molecules.

Then, when a detection liquid is supplied to the well 56 to measure the object to be detected, Id of each of the sensor 3′ and the reference element 4 shifts in response to the pH of the detection liquid thereof. Additionally, Id shifts in the sensor 3′ along with the object to be detected being captured by the probe molecules.

The effects of Id shift due to pH in the sensor 3′ can be corrected by the difference from the reference element 4.

In a sensor using graphene, the object to be detected can be detected with high sensitivity if electric transport characteristics near the Dirac point are used. Meanwhile, there are concerns that the Dirac point may shift due to the measurement atmosphere, concentration of the object to be detected, or the like, and that the insulating film will break when gate voltage is applied near the Dirac point. Furthermore, in regions in which there are sufficient free electrons, there are concerns for a lowering in the rate of change in electric characteristics with respect to the concentration of the object to be detected, and a reduction in decomposability relative to the concentration of the object to be detected.

In the embodiment shown below, a carrier control layer is provided near the graphene film 21 to control the carrier amount in the graphene film 21.

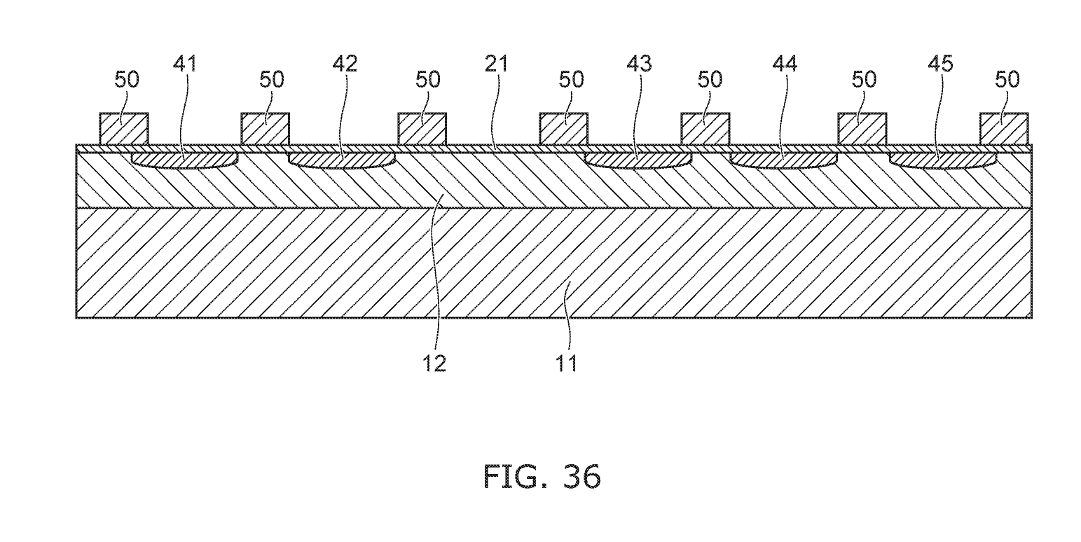

FIG. 36 is a schematic cross-sectional view of a structure provided with a carrier control layer in a sensor using a graphene film.

The foundation film 12 is provided on the substrate 11, and the graphene film 21 is provided on the foundation film 12. The graphene film 21 contacts an electrode 50.

Carrier control layers 41 to 45 are provided on the surface of the foundation film 12, and the graphene film 21 contacts the carrier control layers 41 to 45. The carrier control layers 41 to 45 are provided near the graphene film 21 in a range of, for example, appropriately 5 nm (equivalent to the device length).

The carrier control layers 41 to 45 have a plurality of regions 41 to 45 having different carrier implantation amounts to the graphene film 21. For example, the region 42 has a larger carrier implantation amount to the graphene film 21 than the region 41, the region 43 has a larger carrier implantation amount to the graphene film 21 than the region 42, the region 44 has a larger carrier implantation amount to the graphene film 21 than the region 43, and the region 45 has a larger carrier implantation amount to the graphene film 21 than the region 44.

In a case where the region between the pair of electrodes 50 in FIG. 36 is one element, six elements are illustrated in FIG. 36. From among these six elements, a carrier control layer is not provided on the third element from the left side.

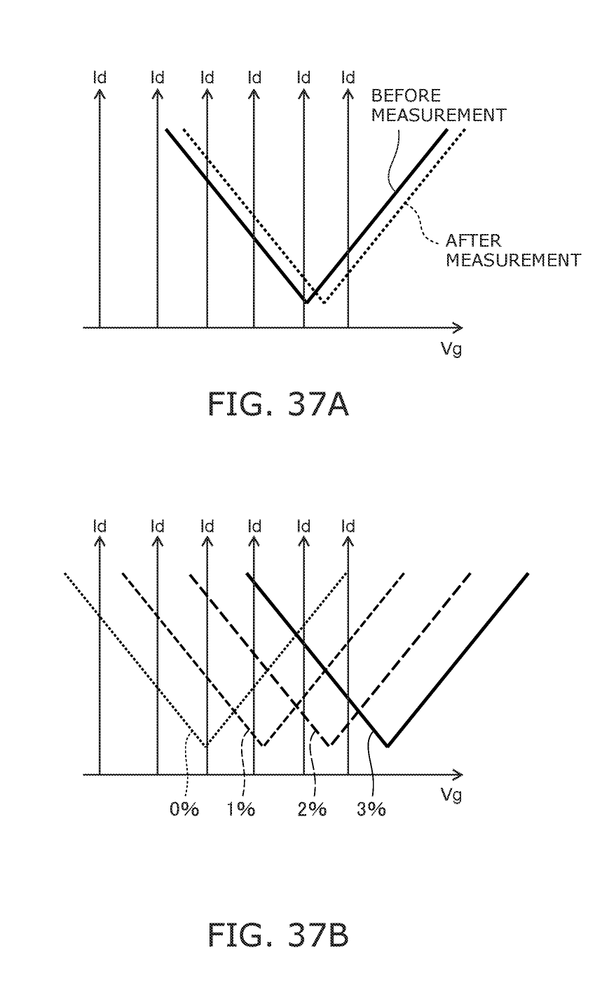

FIG. 37A illustrates the Id-Vg characteristics before measurement (solid line) and the Id-Vg characteristics after measurement (broken line) of the object to be detected in the sensor illustrated in FIG. 36.

The Id axis shifts based on the carrier amount (charge amount) of the six elements in FIG. 36. The leftmost Id axis corresponds to the element having the region 41 in FIG. 36. The second Id axis from the left corresponds to the element having the region 42 in FIG. 36. The third Id axis from the left corresponds to the element that does not have a carrier control layer (third element from the left) in FIG. 36. The fourth Id axis from the left corresponds to the element having the region 43 in FIG. 36. The fifth Id axis from the left corresponds to the element having the region 44 in FIG. 36. The rightmost Id axis corresponds to the element having the region 45 in FIG. 36.

With the element that does not have a carrier control layer (third Id axis from the left in FIG. 37A), the Dirac point cannot be evaluated unless a high gate voltage Vg is applied, but, for example, in a case where evaluation is carried out in the element having the region 44 (second Id axis from the right), the change rate of Id is large even with a low gate voltage Vg. By appropriately selecting the element used for measurement from among the plurality of elements having different carrier amounts (charge amounts), high sensitivity detection can be carried out without being affected by the surrounding environment or concentration of the object to be detected.

FIG. 37B illustrates Id-Vg characteristics due to a difference in concentration of the object to be detected. For example, it illustrates Id-Vg characteristics in which the concentration of the object to be detected is 0%, 1%, 2%, and 3%. Furthermore, the leftmost Id axis corresponds to the element having the region 41 in FIG. 36, similar to FIG. 37A. The second Id axis from the left corresponds to the element having the region 42 in FIG. 36. The third Id axis from the left corresponds to the element that does not have a carrier control layer (third element from the left) in FIG. 36. The fourth Id axis from the left corresponds to the element having the region 43 in FIG. 36. The fifth Id axis from the left corresponds to the element having the region 44 in FIG. 36. The rightmost Id axis corresponds to the element having the region 45 in FIG. 36.

By selecting an evaluating element based on the concentration of the object to be detected, the object to be detected can be measured with high sensitivity across a wide concentration.

The foundation film 12 is, for example, an insulating film, and by implanting dopant in the surface of the foundation film 12 thereof by, for example, an ion implantation method, the carrier control layers 41 to 45 can be formed. After forming the carrier control layers 41 to 45, the graphene film 21 is formed on the foundation film 12. Boron (B) can be used as a dopant that supplies a positive hole, and phosphorous (P) and arsenicum (As) can be used as a dopant that supplies electrons.

Alternatively, the carrier control layers 41 to 45 can be formed on the surface of the foundation film 12 by a —OH terminal process or an Si—O—Si terminal process on the surface of the foundation film 12 of an SiO₂ film or the like.

Alternatively, an organic molecule film can be used as the carrier control layers 41 to 45. For example, by using a plurality of molecule films having different structures, the plurality of regions 41 to 45 can be formed having different carrier implantation amounts to the graphene film 21. Alternatively, the plurality of regions 41 to 45 can be formed having different carrier implantation amounts to the graphene film 21 with a surface single molecule modifier such as a self-assembled monolayer (SAM) having different density.

In a sensor using the graphene film 21 in the embodiment described above, a supermolecule can be provided on the surface of the graphene film 21.

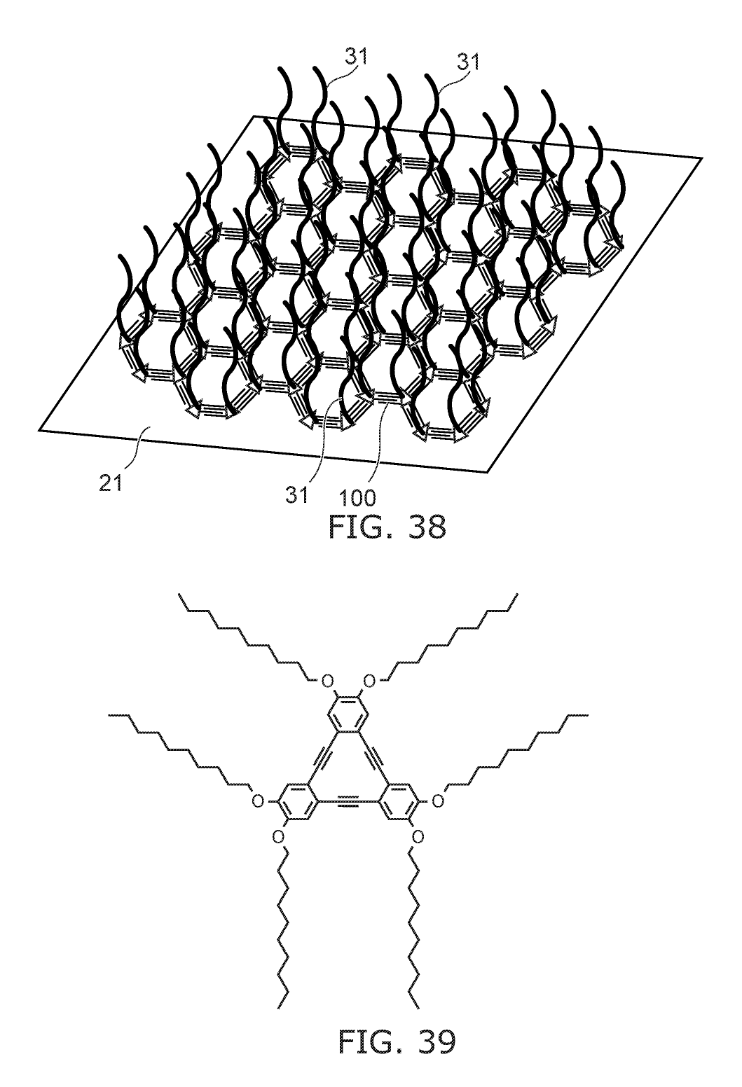

FIG. 38 is a schematic perspective view of a supermolecule 100 provided on the surface of the graphene film 21.

The super molecule 100 is an aggregate of a plurality of a molecule methodically aggregated by coaction between noncovalent molecules, and is disposed in two dimensions on the surface of the graphene film 21. The super molecule 100 can be formed on the surface of the graphene film 21 using, for example, an application method, an evaporation method, or a spray method.

For example, dehydrobenzo [12] annulene (DBA)-OC-based molecules are given as molecules that configure the super molecule 100. DBA-OC-based supermolecules can be formed to be self-aligned on the surface of the graphene film 21 by van der Waals interdigitation.

FIG. 39 is a diagram illustrating the molecular structure of, for example, DBA-OC10. DBA-OC10 has a OC₁₀H₂₁-base.

As illustrated in FIG. 38, probe molecules 31 such as those described above are bonded to one portion of the supermolecule 100. The position or density of the probe molecules 31 are controlled by the supermolecule 100.

For example, by controlling the OC bonding length (number of C) of the DBA-OC-based supermolecule 100, it becomes easy to form the plurality of a probe molecule 31 in high density. This raises the capture probability of the object to be detected, and makes high sensitive analysis possible.

The supermolecule 100 can include at least two types of molecules; first molecules to which the probe molecules 31 bond, and second molecules of a different type than the first molecules, to which the probe molecules do not bond.

A case is possible in which the high density disposing of the probe molecules make the probe molecules inactive, depending on the size of the probe molecules. In such a case, by controlling the ratio of the first molecules and the second molecules in the super molecule 100, the density of the probe molecules 31 can be controlled, and high sensitive detection by high density disposition is possible while maintaining the activity of the probe molecules 31.

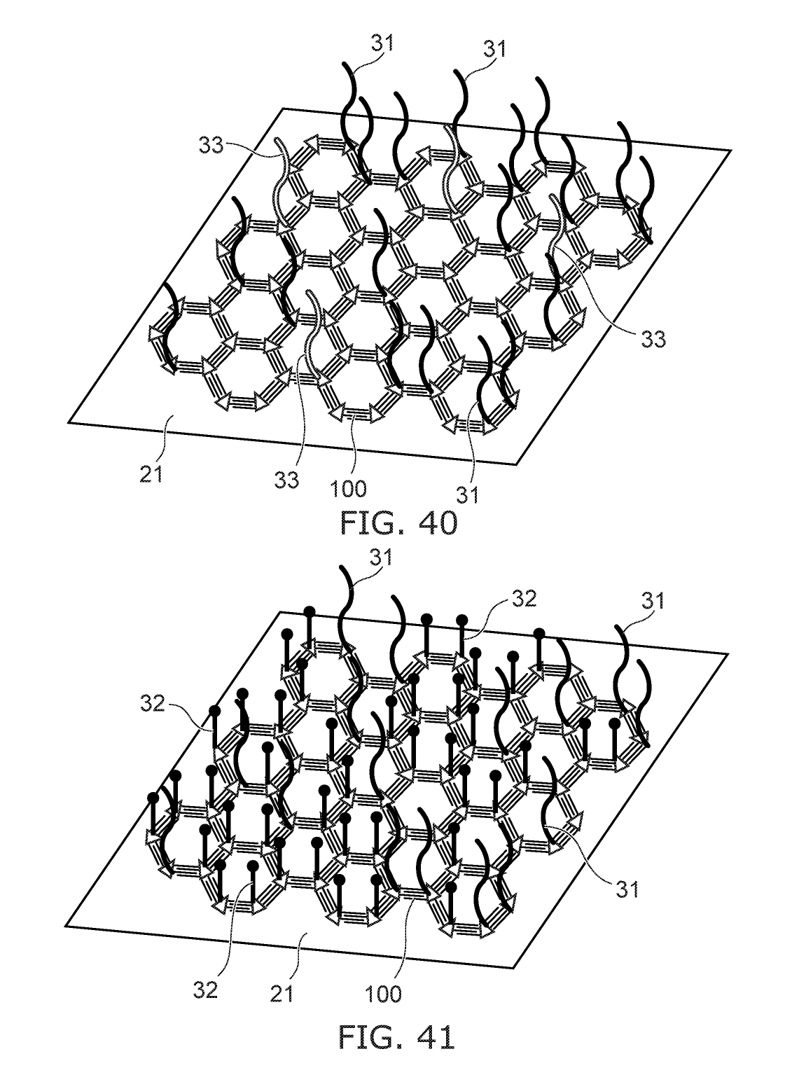

Furthermore, as illustrated in FIG. 40, the probe molecules can include first probe molecules 31, and second probe molecules 33 of a different type than the first probe molecules 31, and the supermolecule 100 can include first molecules that bond with the first probe molecules 31 and second molecules of a different type than the first probe molecules, that bond with the second probe molecules 33.

By controlling the ratio of the first molecules and the second molecules in the super molecule 100, the ratio of the first probe molecules 31 and second probe molecules 33 having different types can be controlled.

Furthermore, as illustrated in FIG. 41, the supermolecule 11 can include first molecules that bond with the probe molecules 31 and second molecules of a different type than the first probe molecules, that bond with noise source blocking molecules (block film) 32.

The noise source blocking molecules 32 are molecules that block substances of different types than the object to be detected (noise source) from approaching the supermolecule 100 or the graphene film 21 by the probe molecules 31, and for example, can use pseudo lipid-based molecules.

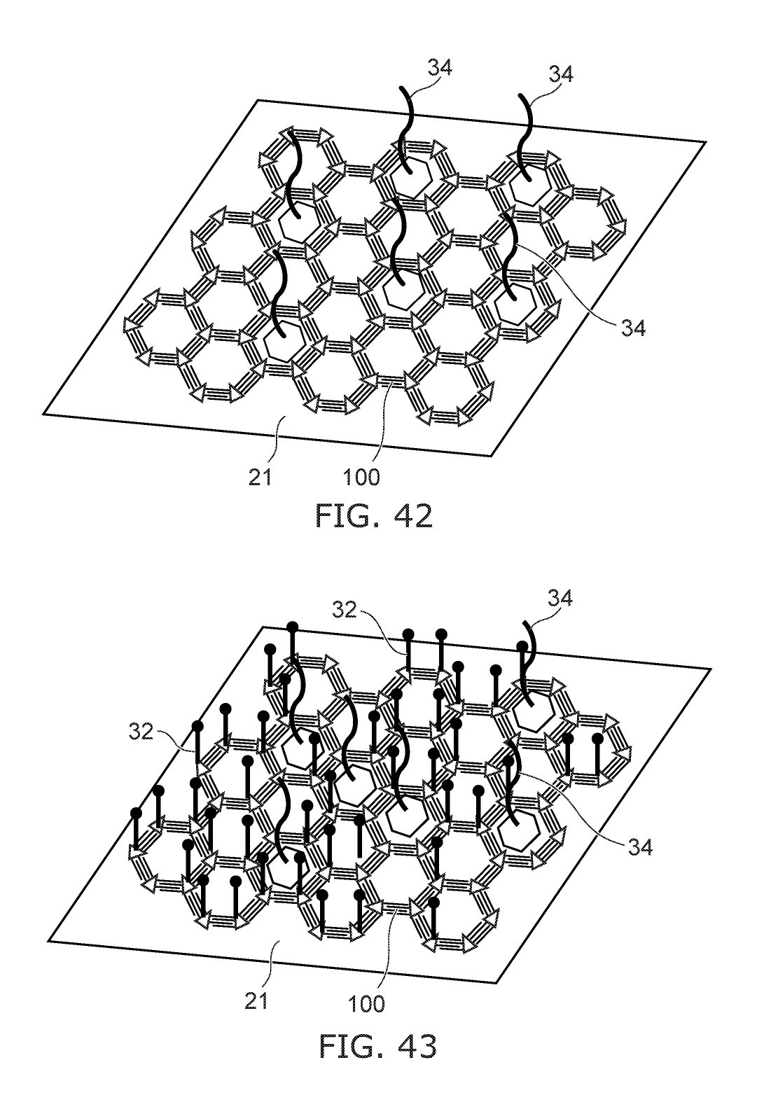

Moreover, as illustrated in FIG. 42, the probe molecules 34 can be disposed in the gap between molecules that configure the supermolecule 100. The probe molecules 34 are bonded to the graphene film 21 via the gaps of the supermolecule 100. The position or density of the probe molecules 34 that bond to the graphene film 21 are controlled by the supermolecule 100.

Moreover, as illustrated in FIG. 43, the probe molecules 34 can be disposed in the gaps of the supermolecule 100, and the noise source blocking molecules 32 can be disposed on the supermolecule 100.

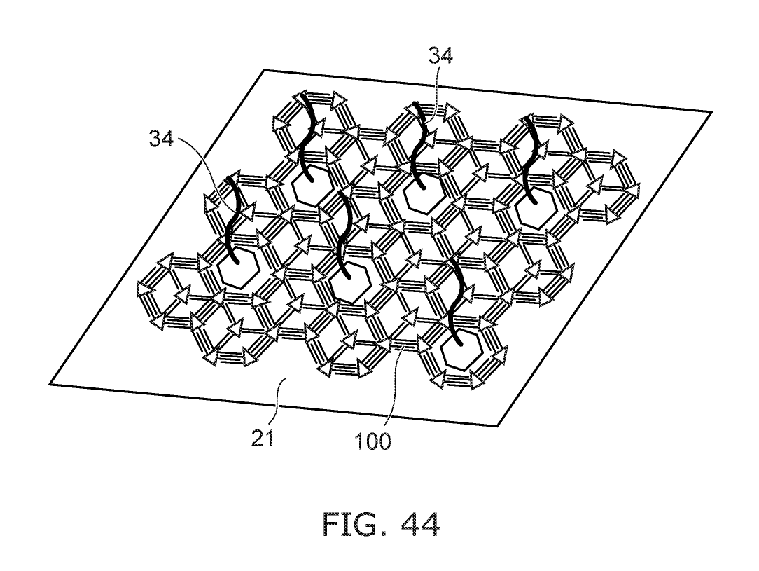

Moreover, as illustrated in FIG. 44, the length of a portion of the molecules configuring the supermolecule 100 can be changed, locations on which the probe molecules 34 can be formed on the supermolecule 100 can be controlled, and the density of the probe molecules 34 can be controlled.

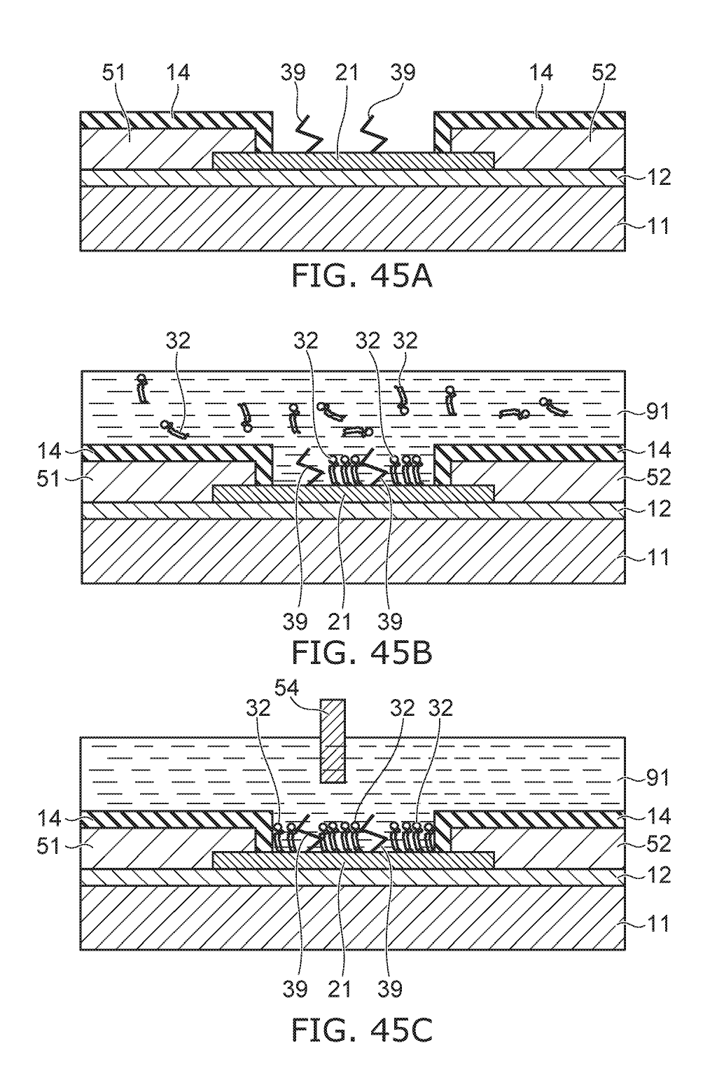

Next, an example of a specific use method of the sensor of the embodiment using the graphene film 21 will be described referring to FIG. 45A to FIG. 45C.

In a state of starting use illustrated in FIG. 45A, linkers 39 are bonded to the graphene film 21, for fixing the probe molecules to the graphene film 21. The linkers 39 are, for example, straight-chain molecules, and for example, a maleimide group is modified on the tip end of the linkers 39.

Next, as illustrated in FIG. 45B, a solution 91 having a blocking agent 32 dispersed therein is supplied on the graphene film 21. The blocking agent 32 is, for example, a phospholipid film. Because graphene is strongly hydrophobic, the phospholipid film rapidly self-organizes on the surface of the graphene film 21.

After the surface of the graphene film 21 is covered by the blocking agent 32, excess blocking agent 32 that floats in the solution 91 is cleaned.

Next, the covering state (coverage) of the blocking agent 32 is monitored. For example, the first electrode (source electrode) 51 and the second electrode (drain electrode 52) are made to have the same potential, and the resistance of the blocking agent (phospholipid) 32 is measured from the difference in potential between these electrodes 51 and 52, and an upper electrode 54 contacting the solution 91. For example, a gigaohm level high resistance is shown when the phospholipid is properly covered, and a megaohm level resistance is shown when there is a covering defect.

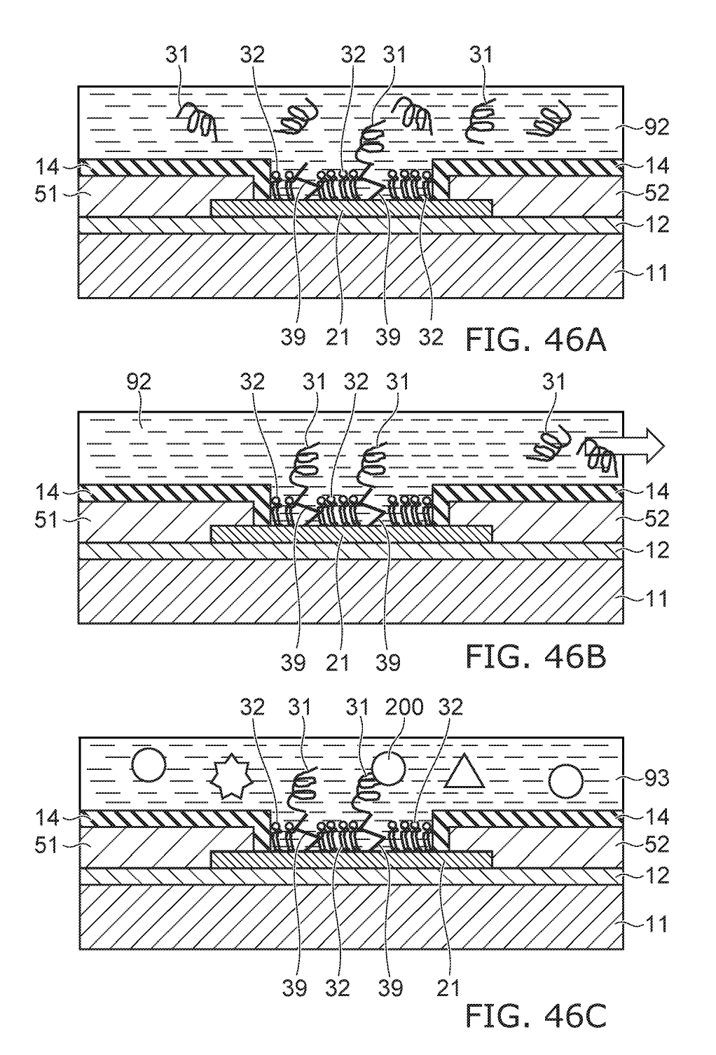

Next, as illustrated in FIG. 46A, a solution 92 having probe molecules 31 dispersed therein is supplied on the graphene film 21. For example, when the tip end of the linkers 39 is modified by maleimide group and the terminal of the probe molecules 31 is modified by thiol group, the probe molecules 31 bond to the linkers 39 due to an addition reaction of maleimide and thiol, and the probe molecules 31 are fixed near the surface of the graphene film 21.

The state of the probe molecules 31 fixed on the graphene film 21 increasing can be monitored by the electric characteristics of graphene FET. Because the charge of the probe molecules 31 affect the graphene film 21 and the fermi level shifts, the current between the first electrode 51 and the second electrode 52 (current between source/drain) changes. When probe molecules 31 are fixed to all of the linkers 39, the change in current between the source/drain saturates.

Next, as illustrated in FIG. 46B, excess probe molecules 31 that are floating in the solution 91 are cleaned. Afterward, as illustrated in FIG. 46C, a detection liquid 93 is supplied on the graphene film 21, and the current between the source/drain is measured.

When a target molecule (object to be detected) 200 exists in the detection liquid 93, because the target molecule 200 is captured by the probe molecules 31 and is fixed near the surface of the graphene film 21, the current between the source/drain fluctuates due to the charge of the target molecule 200. By this, the existence of the target molecule 200 in the detection liquid 93 can be detected.

Furthermore, the higher the concentration of target molecules 200, the more probable one is captured by the probe molecules 31, so the fluctuation of current between the source/drain becomes steep. By this, the concentration of the target molecules 200 in the detection liquid 93 can be detected.

A method called enzyme-linked immuno sorbent assay (ELIZA) is known as a techique for analyzing the concentration of a target molecule in a detection liquid using probe molecules. The process flow can be shortened in the method described above using a graphene sensor for this ELIZA. Furthermore, because the evaluation of each process (quality of the blocking agent or the bonding state of the probe molecules) can be monitored, reproducibility rises because differences caused by the operator do not easily occur.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modification as would fall within the scope and spirit of the inventions.

What is claimed is:

1. A sensor, comprising:
a graphene film having an opening, the opening dominantly having either a zigzag edge or an armchair edge, and
at least two electrodes electrically contacting the graphene film, for reading a change in electric characteristics of the graphene film due to coaction with an object to be detected.

2. The sensor according to claim 1, wherein
an outline of the opening is a polygon, and
the zigzag edge or the armchair edge dominantly appears on a portion corresponding to a side of the polygon.

3. The sensor according to claim 2, wherein the outline of the opening is a hexagon.

4. The sensor according to claim 1, further comprising a probe molecule adsorbed or bonded to the graphene film.

5. A sensor, comprising:
a graphene film having at least one opening;
a probe molecule bonded to an edge of the opening; and
at least two electrodes electrically contacting the graphene film, for reading a change in electric characteristics of the graphene film when association, separation, or reaction occurs between the probe molecule and an object to be detected,
no more than two probe molecules bonding to one of the openings.

6. The sensor according to claim 5, wherein a probe molecule of a different type than the probe molecule bonded to the edge of the opening adsorbs or bonds with a surface of the graphene film.

7. The sensor according to claim 5, wherein the opening has at least one of a zigzag edge and an armchair edge.

8. The sensor according to claim 4, wherein the probe molecule bonds with either one of the zigzag edge and the armchair edge of the opening.

9. The sensor according to claim 8, wherein the probe molecule covalently bonds with the zigzag edge or the armchair edge.

10. The sensor according to claim 8, wherein a probe molecule that blocks bonding of the probe molecule is bonded to the other of the zigzag edge and the armchair edge.

11. The sensor according to claim 4, wherein different types of probe molecules are bonded to each of the zigzag edge and the armchair edge.

12. The sensor according to claim 1, further comprising a protective film that covers an end of the graphene film.

13. A sensor, comprising:
a sensor element electrically detecting change in surface charge;
a liquid material provided on the sensor element and contacting a surface of the sensor element, the liquid material including a probe molecule and a water, the probe molecule having a characteristic of selectively recognizing a specific substance; and
a thin film covering the liquid material, and having a plurality of through-holes.

14. The sensor according to claim 13, wherein the sensor element includes a graphene film.

15. The sensor according to claim 1, further provided with a carrier control layer provided near the graphene film, the carrier control layer controlling a carrier amount in the graphene film.

16. The sensor according to claim 15, wherein the carrier control layer has a plurality of regions of different carrier implantation amounts to the graphene film.

17. The sensor according to claim 15, wherein
the graphene film is provided on a foundation film, and
the carrier control layer is provided on the foundation film.

18. A sensor, comprising:
a graphene film;
a supermolecule provided on a surface of the graphene film;
a probe molecule bonded to at least one of the graphene film and the supermolecule; and
at least two electrodes electrically contacting the graphene film, for reading a change in electric characteristics of the graphene film when association, separation, or reaction occurs between the probe molecule and an object to be detected.

19. The sensor according to claim 18, wherein
the supermolecule comprises:
a first molecule that bonds with the probe molecule; and
a second molecule of a different type than the first molecule, to which the probe molecule does not bond.

20. The sensor according to claim 18, wherein
the probe molecule comprises:
a first probe molecule; and
a second probe molecule of a different type than the first molecule, and
the supermolecule comprises:
a first molecule that bonds with the first probe molecule; and
a second molecule of a different type than the first molecule, that bonds with the second probe molecule.

21. The sensor according to claim 18, wherein
the supermolecule comprises:
a first molecule that bonds with the probe molecule; and
a second molecule of a different type than the first molecule, that bonds with a noise source blocking molecule.
